SEQUENCE LISTING (37 sequences)

SEQ ID NO: 1 - protein, 16 aa, Artificial Sequence
  Linker
AKTTPKLEEGEFSEAR

SEQ ID NO: 2 - protein, 17 aa, Artificial Sequence
  Linker
AKTTPKLEEGEFSEARV

SEQ ID NO: 3 - protein, 9 aa, Artificial Sequence
  Linker
AKTTPKLGG

SEQ ID NO: 4 - protein, 10 aa, Artificial Sequence
  Linker
SAKTTPKLGG

SEQ ID NO: 5 - protein, 17 aa, Artificial Sequence
  Linker
AKTTPKLEEGEFSEARV

SEQ ID NO: 6 - protein, 6 aa, Artificial Sequence
  Linker
SAKTTP

SEQ ID NO: 7 - protein, 10 aa, Artificial Sequence
  Linker
SAKTTPKLGG

SEQ ID NO: 8 - protein, 6 aa, Artificial Sequence
  Linker
RADAAP

SEQ ID NO: 9 - protein, 9 aa, Artificial Sequence
  Linker
RADAAPTVS

SEQ ID NO: 10 - protein, 12 aa, Artificial Sequence
  Linker
RADAAAAGGPGS

SEQ ID NO: 11 - protein, 6 aa, Artificial Sequence
  Linker
SAKTTP

SEQ ID NO: 12 - protein, 10 aa, Artificial Sequence
  Linker
SAKTTPKLGG

SEQ ID NO: 13 - protein, 18 aa, Artificial Sequence
  Linker
SAKTTPKLEEGEFSEARV

SEQ ID NO: 14 - protein, 5 aa, Artificial Sequence
  Linker
ADAAP

SEQ ID NO: 15 - protein, 12 aa, Artificial Sequence
  Linker
ADAAPTVSIFPP

SEQ ID NO: 16 - protein, 5 aa, Artificial Sequence
  Linker
TVAAP

SEQ ID NO: 17 - protein, 12 aa, Artificial Sequence
  Linker
TVAAPSVFIFPP

SEQ ID NO: 18 - protein, 6 aa, Artificial Sequence
  Linker
QPKAAP

SEQ ID NO: 19 - protein, 13 aa, Artificial Sequence
  Linker
QPKAAPSVTLFPP

SEQ ID NO: 20 - protein, 6 aa, Artificial Sequence
  Linker
AKTTPP

SEQ ID NO: 21 - protein, 13 aa, Artificial Sequence
  Linker
AKTTPPSVTPLAP

SEQ ID NO: 22 - protein, 6 aa, Artificial Sequence
  Linker
AKTTAP

SEQ ID NO: 23 - protein, 13 aa, Artificial Sequence
  Linker
AKTTAPSVYPLAP

SEQ ID NO: 24 - protein, 6 aa, Artificial Sequence
  Linker
ASTKGP

SEQ ID NO: 25 - protein, 13 aa, Artificial Sequence
  Linker
ASTKGPSVFPLAP

SEQ ID NO: 26 - protein, 15 aa, Artificial Sequence
  Linker
GGGGSGGGGSGGGGS

SEQ ID NO: 27 - protein, 15 aa, Artificial Sequence
  Linker
GENKVEYAPALMALS

SEQ ID NO: 28 - protein, 15 aa, Artificial Sequence
  Linker
GPAKELTPLKEAKVS

SEQ ID NO: 29 - protein, 15 aa, Artificial Sequence
  Linker
GHEAAAVMQVQYPAS

SEQ ID NO: 30 - protein, 15 aa, Artificial Sequence
  Linker
RADAAAAGGGGSSSS

SEQ ID NO: 31 - protein, 305 aa, Artificial Sequence [sequence omitted]

SEQ ID NO: 32 - protein, 451 aa, Artificial Sequence
  Heavy Chain
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 214 aa, Artificial Sequence
  Light Chain
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 34 - protein, 542 aa, Artificial Sequence
  Heavy Chain
QHHGVTKCNITCSKMTSKIPVALLIHYQQNQASCGKRAIILETRQHRLFCADPKEQWVKDAMQHLDRQAAALTRNGGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 541 aa, Artificial Sequence [sequence omitted]

SEQ ID NO: 36 - protein, 552 aa, Artificial Sequence [sequence omitted]

SEQ ID NO: 37 - protein, 552 aa, Artificial Sequence
  Heavy Chain
QPDAINAPVTCCYNFTNRKISVQRLASYRRITSSKCPKEAVIFKTIVAKEICADPKQKWVQDSMDHLDKQTQTPKTGGGGSGGGGSGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2009/0226435 A1
(43) Pub. Date: Sep. 10, 2009

(54) Engineered fusion molecules immunotherapy in cancer and inflammatory diseases

(76) Inventor: Sanjay Khare, Newbury Park, CA (US)

Correspondence Address: Craig A. Crandall, APC, 3034 Deer Valley Avenue, Newbury Park, CA, 91320, US

(21) Appl. No.: 12/381,058

(22) Filed: Mar. 7, 2009

Related U.S. Application Data
(60) Provisional application No. 61/068,628, filed on Mar. 8, 2008.

(51) Int. Cl. A61K 39/395; C07K 16/30; A61P 35/00

(52) U.S. Cl. 424/133.1; 530/387.3

(57) ABSTRACT

The field of the present invention relates to genetically engineered fusion molecules, methods of making said fusion molecules, and uses thereof in anti-tumor immunotherapies. More specifically, the present invention relates to engineered fusion molecules consisting of a tumor targeting moiety fused with one or more costimulatory molecules/chemokines/cytokines.

### RELATED PATENT APPLICATIONS

This application claims benefit of U.S. Provisional Application No. 61/068,628, filed on Mar. 8, 2008, incorporated in its entirety by reference herein.

### TECHNICAL FIELD

The field of the present invention relates to genetically engineered fusion molecules, methods of making said fusion molecules, and uses thereof in anti-tumor and anti-inflammatory immunotherapies. More specifically, the present invention relates to engineered fusion molecules consisting of a tumor or inflammatory cell targeting moiety fused with one or more costimulatory molecules/cytokines and/or chemokines. Importantly, the engineered fusion molecules of the present invention provide focused immunological action to the disease site, recruitment and activation of effector cytotoxic and NK cells, increased target cell killing mediated by improved ADCC with the possibility of demonstrating efficacy in patients with Fc receptor polymorphism, and enhanced activation of T cells. As such, the novel fusion molecules provide new and more effective immunotherapeutic approaches to a variety of cancer and inflammatory diseases.

### BACKGROUND OF THE INVENTION

Today, cancer remains a major cause of death and various diagnostic and therapeutic methods for cancer have been developed. Immunotherapy is the name given to cancer treatments that use the immune system to attack cancers. Systemic immunotherapy refers to immunotherapy that is used to treat the whole body and is more commonly used than local immunotherapy which is used to treat one “localized” part of the body, particularly when a cancer has spread. Although cancer cells are less immunogenic than pathogens, the immune system is clearly capable of recognizing and eliminating tumor cells, and cancer immunotherapy attempts to harness the exquisite power and specificity of the immune system for treatment of malignancy. Unfortunately, tumors frequently interfere with the development and function of immune responses, i.e., the suppressive milieu present within established tumors inhibits effective immune responses. Thus, the challenge for immunotherapy is to use advances in cellular and molecular immunology to develop strategies which manipulate the local tumor environment to promote a proinflammatory environment, promote dendritic cell activation, and effectively and safely augment anti-tumor responses.

Conventionally, immunotherapy for cancers had previously been centered on nonspecific immunotherapy. In recent years, however, it has been clarified that T cells play an important role in tumor rejection in living bodies. As a result, extensive efforts are now focused on T cell responses and regulators of T cell activation. Targeted destruction of malignancies by enhancing T cell responses is an attractive modality for therapy because it potentially allows for exquisite specificity and potent activity in the elimination of target cells while avoiding toxicities associated with many other standard approaches.

Costimulatory molecules are important regulators of T cell activation and thus are the favored targets for therapeutic manipulation of the immune response. Often, tumors lack costimulatory molecules and therefore cytotoxic response is difficult to generate in vivo (Chen et al., Cell, 71:1093, 1992). In efforts to address and overcome this problem, several antigen-specific cytotoxic T cells mediated therapies have been evaluated. Such therapies include: (a) costimulatory gene transfer to tumors (see, e.g., Friedlander et al., Am. J. Respir. Cell Mol. Biol., 29:321, 2003; Li et al., Cell Mol Immunol., 2:81, 2005); (b) adoptive immunotherapy=ex vivo stimulation of T cells and transfer of antigen specific T cells back to patients (see, e.g., Ho et al., Cancer Cell, 3:431, 2003); and (c) dendritic cells (DC) loading with tumor antigens and related procedures (see, e.g., Morse and Lyerly, Cytokines Cell Mol Ther, 4:35, 1998; Hart and Hill, Immunology and Cell Biology, 77:451, 1999). These efforts have been particularly challenging. For example, as relates specifically to item (a), there is lack of an optimized vector for gene therapy. As relates to items (b) and (c), these therapies are cumbersome due to the individualized nature of the therapy, e.g., ex vivo generation of cytotoxic T cells followed by transfer to each patient and/or tumor antigen loading to DC for each patient.

Besides the antigen-specific cytotoxic T cell mediated therapies above, therapies focused on utilization of depleting antibodies to specific tumor antigens have been explored with great success (see, e.g., reviews by Blattman and Greenberg, Science, 305:200, 2004; Adams and Weiner, Nat Biotech, 23:1147, 2005). A few examples of such tumor antigen-specific, depleting antibodies are Herceptin® (anti-Her2/neu mAb)(Baselga et al., J Clin Oncology, Vol 14:737, 1996; Baselga et al., Cancer Research, 58:2825, 1998; Shak, Semin. Oncology, 26 (Suppl 12):71, 1999; Vogal et al. J Clin Oncology, 20:719, 2002); and Rituxan® (anti-CD20 mAb)(Colombat et al., Blood, 97:101, 2001). Hudziak et al. (U.S. Pat. No. 6,165,464) describe and claim an isolated human antibody which specifically binds to Her2 receptor. These and other tumor antigen-specific, depleting antibody therapies have clearly made a mark in oncology treatment. However, as monotherapy they generally work in about 30% of patients and with partial response. As such, there continues to be extensive research directed toward evaluating and improving the response rates associated with such therapies.

Tumor antigen-specific, depleting antibody therapies deplete tumor cells by antibody-directed cellular cytotoxicity (ADCC) and complement dependent cytotoxicity (CDC). The ADCC is an immune effector mechanism that requires: 1) therapeutic binding to the antigen through antibody CDRs; and 2) antibody Fc binding to Fc receptors (FcR) expressed on natural killer (NK) cells. Though the exact mechanism of ADCC function in not known, two mechanisms have been postulated by scientists: 1) a passive mechanism where FcR on effector cells serve as crosslinking molecules; and 2) an active mechanism whereby activation of effector cells leads to production and release of cytotoxic molecules such as perforin (for pore formation) and granzyme B (for proteolysis). Various defects have been associated with suboptimal response of ADCC. For example, a correlation between the lack of drug response and an FcR mutation has been established in many studies (Cartron et al., Blood, 99:754, 2002); Bowles and Weiner, JIM 304:88, 2005). Additional defects include: (a) lack of NK cells and effector cytotoxic T cells recruitment to the target site; (b) expression of killer inhibitory receptors (KIR) on NK cells; and (c) expression of inhibitory Fc receptor.

Research efforts directed to resolving some of the issues associated with antigen-specific cytotoxic T cells mediated therapies and the tumor antigen-specific, depleting antibody therapies and been extensive. For example, Holzer et. al. (U.S. Pat. No. 5,824,782) describe cancer therapeutic agents comprising an antibody or antibody fragment specific for the human epidermal growth factor receptor linked to a member of the IL-8 chemokine family. The claimed immunoconjugates are shown to induce cytotoxic and chemotactic activity and thus purported to be suitable for targeted tumor therapy in human patients. There is no demonstration of in vivo therapy using the immunoconjugates.

Lazar et al. (U.S. Pat. No. 7,317,091) describe and claim Fc variants that are optimized for their ability to bind Fc gamma receptors as compared to their parent polypeptide. The described Fc variants are generally contained within a variant protein that preferably comprises an antibody or Fc fusion protein. The Fc variants were reported to have significant ADCC improvements.

Epstein et al., (U.S. Patent Application Nos. 20040228836 and 20070141025) disclose cancer therapeutic agents comprising a cancer targeting molecule linked to a liver-expressed cytokine (LEC). The preferred targeting molecule is an antibody specific for a tumor cell-surface antigen, a stromal component of a tumor, or an intracellular antigen. Importantly, only LECs are contemplated for use, and specific LEC SEQ IDs are provided for such use.

The present invention is directed to resolving the issues above by: 1) improving antigen-specific cytotoxic T cells mediated therapies by providing new and improved genetically engineered fusion molecules which provide for focused delivery of a missing co-stimulatory molecule/cytokine/chemokine to the tumor site to promote enhanced recruitment and activation of effector T cells and NK cells; and 2) improving tumor antigen-specific, depleting antibody therapies by providing new and improved genetically engineered fusion molecules having superior activity as compared to currently marketed drugs.

### SUMMARY OF THE INVENTION

The present inventor seeks to improve on current antigen-specific cytotoxic T cells mediated therapies. As such, one aspect of the present invention is to provide a genetically engineered fusion molecule comprising a cell/tumor targeting moiety fused to one or more costimulatory molecules. In one embodiment, the fusion molecule will comprise a tumor targeting moiety and a costimulatory molecule attached to the tumor targeting moiety via a linker as depicted in any of the FIGS. 1, 3 and 5. In another embodiment, the fusion molecule will further comprise a targeting peptide attached to the tumor targeting moiety via a linker as depicted in any of the FIGS. 2, 4, 6 and 7. In other alternative embodiments, the fusion molecule may comprise a costimulatory molecule and a cytokine attached via linkers to the tumor targeting moiety. In particularly preferred embodiments, the targeting moiety will be selected form the group consisting of, but not limited to, a depleting antibody, Fab, Fab2, scFv, tumor binding peptide, or minimalistic tumor/inflammatory cell binding domain; and the costimulatory molecule will be selected from the group consisting of, but not limited to, one or more of B7.1, B7.2, B7RP1, B7h, PD1, PDL1/PDL2, OX40L, CD86, CD40/CD40L or 41BB/41BBL. Importantly, said fusion molecules deliver the missing costimulatory molecule to the tumor site and promote optimal activation of T cells. And because of the nature of the targeting moiety, focused delivery of the signal is expected primarily to the tumor site. With focused delivery and dose optimization, the fusion molecules of the present invention are not expected to cause systemic activation of immune system leading to autoimmunity as seen with some non-antigen specific molecules currently in the clinical trials.

The present inventor also seeks to improve on existing tumor antigen-specific, depleting antibody therapies. As such, another aspect of the present invention is to provide a genetically engineered fusion molecule comprising a cell/tumor targeting moiety fused to a chemokine. In one embodiment, the fusion molecule will comprise a tumor targeting moiety and a chemokine attached to the tumor targeting moiety via a linker as depicted in any of the FIGS. 1, 3 and 5. In another embodiment, the fusion molecule will further comprise a targeting peptide attached to the tumor targeting moiety via a linker as depicted in any of the FIGS. 2, 4, 6 and 7. In other alternative embodiments, the fusion molecule may comprise a chemokine and a cytokine attached via linkers to the tumor targeting moiety. Importantly, said fusion molecules exhibit increased ADCC and enhanced activation of T cells and/or NK cells at the tumor site as compared to said tumor target moiety; addition of the cytokine will serve to further enhance T cell recruitment to increase ADCC and promote optimal activation of effector T cells.

Another aspect of the present invention relates to providing an efficient and convenient method for preparing a genetically engineered fusion molecule of the present invention. The method comprises the steps of: 1) preparing/obtaining a cell/tumor targeting moiety; 2) preparing/obtaining a costimulatory molecule and/or a chemokine and/or cytokine; 3) preparing/obtaining a linker; 4) attaching 1) to 2) using said linker to prepare a fusion molecule; and 5) purifying said fusion molecule. Alternatively, the method may comprise, after step 4), step 5) preparing/obtaining a targeting peptide; step 6) preparing/obtaining a second linker; step 7) attaching the targeting peptide of step 5) to the fusion molecule of step 4) using said second linker to prepare a fusion molecule; and 8) purifying said fusion molecule.

Another aspect of the present invention relates to a pharmaceutical composition, and method of preparing said pharmaceutical composition, wherein said composition comprises the genetically engineered fusion molecule of the present invention as an active ingredient, in a pharmaceutically acceptable carrier.

Another aspect of the present invention relates to methods of therapeutically treating a disease state in a subject. Such methods include administering an effective amount of a genetically engineered fusion molecule of the present invention in pharmaceutically acceptable carrier to the subject, wherein such administration elicits an immune response in a subject.

Another aspect of the present invention relates to a method of treating tumors or tumor metastases in a patient, comprising administering to said patient a therapeutically effective amount of a genetically engineered fusion molecule of the present invention in pharmaceutically acceptable carrier, wherein such administration promotes tumor regression and/or tumor death.

### BRIEF DESCRIPTION OF THE DRAWINGS

FIG. 1 depicts one proposed design for a genetically engineered fusion molecule of the present invention. In FIG. 1, the ovals labeled as VL, VH, CL, CH1, CH2 and CH3 represent an example wherein the tumor targeting agent is in the form of a full length antibody as defined herein. The oval labeled C represents a cytokine, chemokine, or costimulatory molecule. A linker is represented by the squiggled line. As depicted in FIG. 1, C is attached to the tumor targeting agent via a linker at the two VH sites. In one alternative embodiment, C will be attached to the tumor targeting agent via a linker at the two VL sites rather than the two VH sites. In yet another alternative, C will be attached to the tumor targeting agent via a linker at the two CH3 sites rather than two VL or two VH sites. Also contemplated are fusion molecules wherein more than one C is attached to the targeting agent.

FIG. 2 depicts another proposed design for a genetically engineered fusion molecule of the present invention. As in FIG. 1, the ovals labeled as VL, VH, CL, CH1, CH2 and CH3 represent an example wherein the tumor targeting agent is in the form of a full length antibody as defined herein. The oval labeled C represents a cytokine, chemokine, or costimulatory molecule, and a linker is represented by the squiggled line. Further depicted in FIG. 2 are a targeting peptide (half circle/arc) and a second linker (straight line). As depicted in FIG. 2, the C is attached to the tumor targeting agent via a linker at the two VH sites and the targeting peptide is attached via a linker at the two VL sites. In one alternative embodiment, C will be attached to the tumor targeting agent via a linker at the two VL sites and the targeting peptide attached via a linker at the two VH sites. In yet another alternative, C will be attached to the tumor targeting agent via a linker at the two VH sites and the targeting peptide attached via a linker at the two CH3 sites. In these designs, it is contemplated that the same linker may be used for the C attachment and the targeting peptide attachment.

FIG. 3 depicts another proposed design for a genetically engineered fusion molecule of the present invention. In FIG. 3, the ovals labeled as VL, VH, CH, CH1, and CH2 represent an example wherein the tumor targeting agent is in the form of a Fab2 as defined herein. The oval label C represents a cytokine, chemokine, or costimulatory molecule. A linker is represented by the squiggled line. As depicted in FIG. 3, C is attached to the tumor targeting agent via a linker at the two VH sites. In one alternative embodiment, C will be attached to the tumor targeting agent via a linker at the two VL sites rather than the two VH sites. In yet another alternative, C will be attached to the tumor targeting agent via a linker at the two CH2 sites rather than two VL or two VH sites. Also contemplated are fusion molecules wherein more than one C is attached to the targeting agent.

FIG. 4 depicts another proposed design for a genetically engineered fusion molecule of the present invention. As in FIG. 3, the ovals labeled as VL, VH, CL, CH1, and CH2 represent an example wherein the tumor targeting agent is in the form of a Fab2 as defined herein. The oval label C represents a cytokine, chemokine, or costimulatory molecule, and the linker is represented by the squiggled line. Further depicted in FIG. 4 are a targeting peptide (half circle/arc) and a second linker (straight line). As depicted in FIG. 4, the C is attached to the tumor targeting agent via a linker at the two VH sites and the targeting peptide is attached via a linker at the two VL sites. In one alternative embodiment, C will be attached to the tumor targeting agent via a linker at the two VL sites and the targeting peptide attached via a linker at the two VH sites. In yet another alternative, C will be attached to the tumor targeting agent via a linker at the two VH sites and the targeting peptide attached via a linker at the two CH2 sites. In these designs, it is contemplated that the same linker may be used for the C attachment and the targeting peptide attachment.

FIG. 5 depicts another proposed design for a genetically engineered fusion molecule of the present invention. In FIG. 5, the ovals labeled as VL, VH, CL, and CH1 represent an example wherein the tumor targeting agent is in the form of a Fab as defined herein. The oval label C represents a cytokine, chemokine, or costimulatory molecule. A linker is represented by the squiggled line. As depicted in FIG. 5, C is attached to the tumor targeting agent via a linker at the VH site. In one alternative embodiment, C will be attached to the tumor targeting agent via a linker at the VL site. In yet another alternative, C will be attached to the tumor targeting agent via a linker at the CH1 site. Also contemplated are fusion molecules wherein more than one C is attached to the targeting agent.

FIG. 6 depicts another proposed design for a genetically engineered fusion molecule of the present invention. As in FIG. 5, the ovals labeled as VL, VH, CL, and CH1 represent an example wherein the tumor targeting agent is in the form of a Fab as defined herein. The oval label C represents a cytokine, chemokine, or costimulatory molecule. A linker is represented by the squiggled line. Further depicted in FIG. 6 are a targeting peptide (half circle/arc) and a second linker (straight line). As depicted in FIG. 6, C is attached to the tumor targeting agent via a linker at the VH site and the targeting peptide attached via a linker at the VL site. In one alternative embodiment, C will be attached to the tumor targeting agent via a linker at the VL site and the targeting peptide attached via a linker at the VH site. In yet another alternative, C will be attached to the tumor targeting agent via a linker at the VH site and the targeting peptide attached via a linker at the CH1 site. In these designs, it is contemplated that the same linker may be used for the C attachment and the targeting peptide attachment.

FIG. 7 depicts another proposed design for a genetically engineered fusion molecule of the present invention. In FIG. 7, the ovals labeled as CH2 and CH3 represent an example wherein the tumor targeting agent is in the form of a peptide as defined herein. The oval label C represents a cytokine, chemokine, or costimulatory molecule. A linker is represented by the squiggled line. Further depicted in FIG. 7 are a targeting peptide (half circle/arc) and a second linker (straight line). As depicted in FIG. 7, C is attached to the tumor targeting agent via a linker at the CH2 site and the targeting peptide attached via a linker at the CH3 site. In another alternative, C will be attached to the tumor targeting agent via a linker at the CH3 site and the targeting peptide attached via a linker at the CH2 site. Alternatively, the tumor targeting peptide may be linked to human serum albumin (HAS). In these designs, it is contemplated that the same linker may be used for the C attachment and the targeting peptide attachment.

### DETAILED DESCRIPTION OF THE INVENTION

As those in the art will appreciate, the foregoing detailed description describes certain preferred embodiments of the invention in detail, and is thus only representative and does not depict the actual scope of the invention. Before describing the present invention in detail, it is understood that the invention is not limited to the particular aspects and embodiments described, as these may vary. It is also to be understood that the terminology used herein is for the purpose of describing particular embodiments only, and is not intended to limit the scope of the invention defined by the appended claims.

As used herein, an “antibody” refers to a protein comprising one or more polypeptides substantially or partially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. A typical immunoglobulin (e.g., antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (VH) refer to these light and heavy chains, respectively.

In a full-length antibody, each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. Immunoglobulin molecules can be of any type (e.g., IgG, IgE, IgM, IgD, IgA and IgY), class (e.g., IgG1, IgG2, IgG 3, IgG4, IgA1 and IgA2) or subclass.

The term “Fc region” is used to define the C-terminal region of an immunoglobulin heavy chain, which may be generated by papain digestion of an intact antibody. The Fc region may be a native sequence Fc region or a variant Fc region. The Fc region of an immunoglobulin generally comprises two constant domains, a CH2 domain and a CH3 domain, and optionally comprises a CH4 domain. Replacements of amino acid residues in the Fc portion to alter antibody effector function are known in the art (see, e.g., Winter, et al., U.S. Pat. Nos. 5,648,260; 5,624,821). The Fc portion of an antibody mediates several important effector functions e.g. cytokine induction, ADCC, phagocytosis, complement dependent cytotoxicity (CDC) and half-life/clearance rate of antibody and antigen-antibody complexes.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigen. Furthermore, in contrast to polyclonal antibody preparations that typically include different antibodies directed against different determinants (epitopes), each monoclonal antibody is directed against a single determinant on the antigen. The modifier “monoclonal” is not to be construed as requiring production of the antibody by any particular method.

The term “human antibody”, as used herein, is intended to include antibodies having variable and constant regions derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include antibodies in which CDR sequences derived from the germline of another mammalian species, such as a mouse, have been grafted onto human framework sequences.

The term “recombinant human antibody”, as used herein, is intended to include all human antibodies that are prepared, expressed, created or isolated by recombinant means, such as antibodies expressed using a recombinant expression vector transfected into a host cell; antibodies isolated from a recombinant, combinatorial human antibody library; antibodies isolated from an animal (e.g., a mouse) that is transgenic for human immunoglobulin genes; or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo. All such recombinant means are well known to those of ordinary skill in the art.

The present invention relates to genetically engineered fusion molecules comprising at least one tumor targeting moiety linked to at least one costimulatory molecule (or at least one chemokine or cytokine) formed through genetic fusion or chemical coupling. By “linked” we mean that the first and second sequences are associated such that the second sequence is able to be transported by the first sequence to a target cell, i.e., fusion molecules in which the tumor targeting moiety is linked to a costimulatory molecule (or chemokine or cytokine) via their polypeptide backbones through genetic expression of a DNA molecule encoding these proteins, directly synthesized proteins, and coupled proteins in which pre-formed sequences are associated by a cross-linking agent. In one embodiment the tumor targeting moiety and costimulatory molecules (or chemokine or cytokine) are linked directly to each other using recombinant DNA techniques. In another embodiment, the tumor targeting moiety and costimulatory molecules (or chemokine or cytokine) are linked via a linker sequence. The term “attached” as used herein refers to such linkages/fusions.

The term “linker” is used to denote polypeptides comprising two or more amino acid residues joined by peptide bonds and are used to link the tumor targeting moiety and costimulatory molecules of the present invention. Such linker polypeptides are well known in the art (see e.g., Holliger, P., et al., Proc. Natl. Acad. Sci. USA, 90:6444, 1993; Poljak, R. J., et al., Structure, 2:1121, 1994). Preferred linkers include, but are not limited to, AKTTPKLEEGEFSEAR (SEQ ID NO: 1); AKTTPKLEEGEFSEARV (SEQ ID NO:2); AKTTPKLGG (SEQ ID NO:3); SAKTTPKLGG (SEQ ID NO:4); AKTTPKLEEGEFSEARV (SEQ ID NO:5); SAKTTP (SEQ ID NO:6); SAKTTPKLGG (SEQ ID NO:7); RADAAP (SEQ ID NO:8); RADAAPTVS (SEQ ID NO:9); RADAAAAGGPGS (SEQ ID NO:10); SAKTTP (SEQ ID NO:11); SAKTTPKLGG (SEQ ID NO:12); SAKTTPKLEEGEFSEARV (SEQ ID NO: 13); ADAAP (SEQ ID NO: 14); ADAAPTVSIFPP (SEQ ID NO: 15); TVAAP (SEQ ID NO:16); TVAAPSVFIFPP (SEQ ID NO:17); QPKAAP (SEQ ID NO:18); QPKAAPSVTLFPP (SEQ ID NO:19); AKTTPP (SEQ ID NO:20); AKTTPPSVTPLAP (SEQ ID NO:21); AKTTAP (SEQ ID NO:22); AKTTAPSVYPLAP (SEQ ID NO:23); ASTKGP (SEQ ID NO:24); ASTKGPSVFPLAP (SEQ ID NO:25); GGGGSGGGGSGGGGS (SEQ ID NO:26); GENKVEYAPALMALS (SEQ ID NO:27); GPAKELTPLKEAKVS (SEQ ID NO:28); GHEAAAVMQVQYPAS (SEQ ID NO:29); and RADAAAAGGGGSSSS (SEQ ID NO:30). The choice of linker sequences is based on crystal structure analysis of several Fab molecules. There is a natural flexible linkage between the variable domain and the CH1/CL constant domain in Fab or antibody molecular structure. This natural linkage comprises approximately 10-12 amino acid residues, contributed by 4-6 residues from C-terminus of V domain and 4-6 residues from the N-terminus of CL/CH1 domain. The N-terminal residues of CL or CH1 domains, particularly the first 5-6 amino acid residues, adopt a loop conformation without strong secondary structures, therefore can act as flexible linkers between the two variable domains. The N-terminal residues of CL or CH1 domains are natural extension of the variable domains, as they are part of the Ig sequences, therefore minimize to a large extent any immunogenicity potentially arising from the linkers and junctions. Linker length contemplated for use can vary from about 5 to 200 amino acids.

The terms “tumor targeting moiety” and “tumor targeting agent”, as used herein, are intended to include any molecule having specificity to a tumor antigen or specificity to a molecule overexpressed in a pathological state. Virtually any antigen may be targeted by the molecules of the present invention, including but not limited to proteins, subunits, domains, motifs, and/or epitopes belonging to the following list of targets: 17-IA, 4-1 BB, 4Dc, 6-keto-PGF1a, 8-iso-PGF2a, 8-oxo-dG, A1 Adenosine Receptor, A33, ACE, ACE-2, Activin, Activin A, Activin AB, Activin B, Activin C, Activin RIA, Activin RIA ALK-2, Activin RIB ALK-4, Activin RIIA, Activin RIIB, ADAM, ADAM10, ADAM12, ADAM15, ADAM17/TACE, ADAM8, ADAM9, ADAMTS, ADAMTS4, ADAMTS5, Addressins, aFGF, ALCAM, ALK, ALK-1, ALK-7, alpha-1-antitrypsin, alpha-V/beta-1 antagonist, ANG, Ang, APAF-1, APE, APJ, APP, APRIL, AR, ARC, ART, Artemin, anti-Id, ASPARTIC, Atrial natriuretic factor, av/b3 integrin, Ax1, b2M, B7-1, B7-2, B7-H, B-lymphocyte Stimulator (BlyS), BACE, BACE-1, Bad, BAFF, BAFF-R, Bag-1, BAK, Bax, BCA-1, BCAM, Bcl, BCMA, BDNF, b-ECGF, bFGF, BID, Bik, BIM, BLC, BL-CAM, BLK, BMP, BMP-2 BMP-2a, BMP-3 Osteogenin, BMP-4 BMP-2b, BMP-5, BMP-6 Vgr-1, BMP-7 (OP-1), BMP-8 (BMP-8a, OP-2), BMPR, BMPR-IA (ALK-3), BMPR-IB (ALK-6), BRK-2, RPK-1, BMPR-II (BRK-3), BMPs, b-NGF, BOK, Bombesin, Bone-derived neurotrophic factor, BPDE, BPDE-DNA, BTC, complement factor 3 (C3), C3a, C4, C5, C5a, C10, CA125, CAD-8, Calcitonin, cAMP, carcinoembryonic antigen (CEA), carcinoma-associated antigen, Cathepsin A, Cathepsin B, Cathepsin C/DPPI, Cathepsin D, Cathepsin E, Cathepsin H, Cathepsin L, Cathepsin O, Cathepsin S, Cathepsin V, Cathepsin X/ZIP, CBL, CCI, CCK2, CCL, CCL1, CCL11, CCL12, CCL13, CCL14, CCL15, CCL16, CCL17, CCL18, CCL19, CCL2, CCL20, CCL21, CCL22, CCL23, CCL24, CCL25, CCL26, CCL27, CCL28, CCL3, CCL4, CCL5, CCL6, CCL7, CCL8, CCL9/10, CCR, CCR1, CCR10, CCR10, CCR2, CCR3, CCR4, CCR5, CCR6, CCR7, CCR8, CCR9, CD1, CD2, CD3, CD3E, CD4, CD5, CD6, CD7, CD8, CD10, CD11a, CD11b, CD11c, CD13, CD14, CD15, CD16, CD18, CD19, CD20, CD21, CD22, CD23, CD25, CD27L, CD28, CD29, CD30, CD30L, CD32, CD33 (p67 proteins), CD34, CD38, CD40, CD40L, CD44, CD45, CD46, CD49a, CD52, CD54, CD55, CD56, CD61, CD64, CD66e, CD74, CD80 (B7-1), CD86, CD89, CD95, CD123, CD137, CD138, CD140a, CD146, CD147, CD148, CD152, CD164, CEACAM5, CFTR, cGMP, CINC, Clostridium botulinum toxin, Clostridium perfringens toxin, CKb8-1, CLC, CMV, CMV UL, CNTF, CNTN-1, COX, C-Ret, CRG-2, CT-1, CTACK, CTGF, CTLA-4, CX3CL1, CX3CR1, CXCL, CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CXCR, CXCR1, CXCR2, CXCR3, CXCR4, CXCR5, CXCR6, cytokeratin tumor-associated antigen, DAN, DCC, DcR3, DC-SIGN, Decay accelerating factor, des(1-3)-IGF-I (brain IGF-1), Dhh, digoxin, DNAM-1, Dnase, Dpp, DPPIV/CD26, Dtk, ECAD, EDA, EDA-A1, EDA-A2, EDAR, EGF, EGFR (ErbB-1), EMA, EMMPRIN, ENA, endothelin receptor, Enkephalinase, eNOS, Eot, eotaxin1, EpCAM, Ephrin B2/EphB4, EPO, ERCC, E-selectin, ET-1, Factor IIa, Factor VII, Factor VIIIc, Factor IX, fibroblast activation protein (FAP), Fas, FcR1, FEN-1, Ferritin, FGF, FGF-19, FGF-2, FGF3, FGF-8, FGFR, FGFR-3, Fibrin, FL, FLIP, Flt-3, Flt-4, Follicle stimulating hormone, Fractalkine, FZD1, FZD2, FZD3, FZD4, FZD5, FZD6, FZD7, FZD8, FZD9, FZD10, G250, Gas 6, GCP-2, GCSF, GD2, GD3, GDF, GDF-1, GDF-3 (Vgr-2), GDF-5 (BMP-14, CDMP-1), GDF-6 (BMP-13, CDMP-2), GDF-7 (BMP-12, CDMP-3), GDF-8 (Myostatin), GDF-9, GDF-15 (MIC-1), GDNF, GDNF, GFAP, GFRa-1, GFR-alpha1, GFR-alpha2, GFR-alpha3, GITR, Glucagon, Glut 4, glycoprotein IIb/IIIa (GP IIb/IIIa), GM-CSF, gp130, gp72, GRO, Growth hormone releasing factor, Hapten (NP-cap or NIP-cap), HB-EGF, HCC, HCMV gB envelope glycoprotein, HCMV) gH envelope glycoprotein, HCMV UL, Hemopoietic growth factor (HGF), Hep B gp120, heparanase, Her2, Her2/neu (ErbB-2), Her3 (ErbB-3), Her4 (ErbB-4), herpes simplex virus (HSV) gB glycoprotein, HSV gD glycoprotein, HGFA, High molecular weight melanoma-associated antigen (HMW-MAA), HIV gp120, HIV IIIB gp120 V3 loop, HLA, HLA-DR, HM1.24, HMFG PEM, HRG, Hrk, human cardiac myosin, human cytomegalovirus (HCMV), human growth hormone (HGH), HVEM, I-309, IAP, ICAM, ICAM-1, ICAM-3, ICE, ICOS, IFNg, Ig, IgA receptor, IgE, IGF, IGF genetically engineered fusion molecules, IGF-1R, IGFBP, IGF-I, IGF-II, IL, IL-1, IL-1R, IL-2, IL-2R, IL-4, IL-4R, IL-5, IL-5R, IL-6, IL-6R, IL-8, IL-9, IL-10, IL-12, IL-13, IL-15, IL-18, IL-18R, IL-23, interferon (INF)-alpha, INF-beta, INF-gamma, Inhibin, iNOS, Insulin A-chain, Insulin B-chain, Insulin-like growth factor 1, integrin alpha2, integrin alpha3, integrin alpha4, integrin alpha4/beta1, integrin alpha4/beta7, integrin alpha5 (alphaV), integrin alpha5/beta1, integrin alpha5/beta3, integrin alpha6, integrin beta1, integrin beta2, interferon gamma, IP-10, I-TAC, JE, Kallikrein 2, Kallikrein 5, Kallikrein 6, Kallikrein 11, Kallikrein 12, Kallikrein 14, Kallikrein 15, Kallikrein L1, Kallikrein L2, Kallikrein L3, Kallikrein L4, KC, KDR, Keratinocyte Growth Factor (KGF), laminin 5, LAMP, LAP, LAP (TGF-1), Latent TGF-1, Latent TGF-1 bp1, LBP, LDGF, LECT2, Lefty, Lewis-Y antigen, Lewis-Y related antigen, LFA-1, LFA-3, Lfo, LIF, LIGHT, lipoproteins, LIX, LKN, Lptn, L-Selectin, LT-a, LT-b, LTB4, LTBP-1, Lung surfactant, Luteinizing hormone, Lymphotoxin Beta Receptor, Mac-1, MAdCAM, MAG, MAP2, MARC, MCAM, MCAM, MCK-2, MCP, M-CSF, MDC, Mer, METALLOPROTEASES, MGDF receptor, MGMT, MHC (HLA-DR), MIF, MIG, MIP, MIP-1-alpha, MK, MMAC1, MMP, MMP-1, MMP-10, MMP-11, MMP-12, MMP-13, MMP-14, MMP-15, MMP-2, MMP-24, MMP-3, MMP-7, MMP-8, MMP-9, MPIF, Mpo, MSK, MSP, mucin (Muc1), MUC18, Muellerian-inhibitin substance, Mug, MuSK, NAIP, NAP, NCAD, N-Cadherin, NCA 90, NCAM, NCAM, Neprilysin, Neurotrophin-3, -4, or -6, Neurturin, Neuronal growth factor (NGF), NGFR, NGF-beta, nNOS, NO, NOS, Npn, NRG-3, NT, NTN, OB, OGG1, OPG, OPN, OSM, OX40L, OX40R, p150, p95, PADPr, Parathyroid hormone, PARC, PARP, PBR, PBSF, PCAD, P-Cadherin, PCNA, PDGF, PDGF, PDK-1, PECAM, PEM, PF4, PGE, PGF, PGI2, PGJ2, PIN, PLA2, placental alkaline phosphatase (PLAP), PIGF, PLP, PP14, Proinsulin, Prorelaxin, Protein C, PS, PSA, PSCA, prostate specific membrane antigen (PSMA), PTEN, PTHrp, Ptk, PTN, R51, RANK, RANKL, RANTES, RANTES, Relaxin A-chain, Relaxin B-chain, renin, respiratory syncytial virus (RSV) F, RSV Fgp, Ret, Rheumatoid factors, RLIP76, RPA2, RSK, S100, SCF/KL, SDF-1, SERINE, Serum albumin, sFRP-3, Shh, SIGIRR, SK-1, SLAM, SLPI, SMAC, SMDF, SMOH, SOD, SPARC, Stat, STEAP, STEAP-II, TACE, TACI, TAG-72 (tumor-associated glycoprotein-72), TARC, TCA-3, T-cell receptors (e.g., T-cell receptor alpha/beta), TdT, TECK, TEM1, TEM5, TEM7, TEM8, TERT, testicular PLAP-like alkaline phosphatase, TfR, TGF, TGF-alpha, TGF-beta, TGF-beta Pan Specific, TGF-beta RI (ALK-5), TGF-beta RII, TGF-beta RIIb, TGF-beta RIII, TGF-beta1, TGF-beta2, TGF-beta3, TGF-beta4, TGF-beta5, Thrombin, Thymus Ck-1, Thyroid stimulating hormone, Tie, TIMP, TIQ, Tissue Factor, TMEFF2, Tmpo, TMPRSS2, TNF, TNF-alpha, TNF-alpha beta, TNF-beta2, TNFc, TNF-RI, TNF-RII, TNFRSF10A (TRAIL R1 Apo-2, DR4), TNFRSF10B (TRAIL R2 DR5, KILLER, TRICK-2A, TRICK-B), TNFRSF10C (TRAIL R3DcR1, LIT, TRID), TNFRSF10D (TRAIL R4 DcR2, TRUNDD), TNFRSF11A (RANK ODF R, TRANCE R), TNFRSF11B (OPG OCIF, TR1), TNFRSF12 (TWEAK R FN14), TNFRSF13B (TACI), TNFRSF13C (BAFF R), TNFRSF14 (HVEM ATAR, HveA, LIGHT R, TR2), TNFRSF16 (NGFR p75NTR), TNFRSF17 (BCMA), TNFRSF18 (GITR AITR), TNFRSF19 (TROY TAJ, TRADE), TNFRSF19L (RELT), TNFRSF1A (TNF RI CD120a, p55-60), TNFRSF1B (TNF RII CD120b, p75-80), TNFRSF26 (TNFRH3), TNFRSF3 (LTbR TNF RIII, TNFC R), TNFRSF4 (OX40 ACT35, TXGP1 R), TNFRSF5 (CD40 p50), TNFRSF6 (Fas Apo-1, APT1, CD95), TNFRSF6B (DcR3 M68, TR6), TNFRSF7 (CD27), TNFRSF8 (CD30), TNFRSF9 (4-1BB CD137, ILA), TNFRSF21 (DR6), TNFRSF22 (DcTRAIL R2 TNFRH2), TNFRST23 (DcTRAIL R1 TNFRH1), TNFRSF25 (DR3 Apo-3, LARD, TR-3, TRAMP, WSL-1), TNFSF10 (TRAIL Apo-2 Ligand, TL2), TNFSF11 (TRANCE/RANK Ligand ODF, OPG Ligand), TNFSF12 (TWEAK Apo-3 Ligand, DR3 Ligand), TNFSF13 (APRIL TALL2), TNFSF13B (BAFF BLYS, TALL1, THANK, TNFSF20), TNFSF14 (LIGHT HVEM Ligand, LTg), TNFSF15 (TL1A/VEGI), TNFSF18 (GITR Ligand AITR Ligand, TL6), TNFSF1A (TNF-a Conectin, DIF, TNFSF2), TNFSF1B (TNF-b LTa, TNFSF1), TNFSF3 (LTb TNFC, p33), TNFSF4 (OX40 Ligand gp34, TXGP1), TNFSF5 (CD40 Ligand CD154, gp39, HIGM1, IMD3, TRAP), TNFSF6 (Fas Ligand Apo-1 Ligand, APT1 Ligand), TNFSF7 (CD27 Ligand CD70), TNFSF8 (CD30 Ligand CD153), TNFSF9 (4-1BB Ligand CD137 Ligand), TP-1, t-PA, Tpo, TRAIL, TRAIL R, TRAIL-R1, TRAIL-R2, TRANCE, transferring receptor, TRF, Trk, TROP-2, TSG, TSLP, tumor-associated antigen CA 125, tumor-associated antigen expressing Lewis Y related carbohydrate, TWEAK, TXB2, Ung, uPAR, uPAR-1, Urokinase, VCAM, VCAM-1, VECAD, VE-Cadherin, VE-cadherin-2, VEFGR-1 (flt-1), VEGF, VEGFR, VEGFR-3 (flt-4), VEGI, VIM, Viral antigens, VLA, VLA-1, VLA-4, VNR integrin, von Willebrands factor, WIF-1, WNT1, WNT2, WNT2B/13, WNT3, WNT3A, WNT4, WNT5A, WNT5B, WNT6, WNT7A, WNT7B, WNT8A, WNT8B, WNT9A, WNT9A, WNT9B, WNT10A, WNT10B, WNT11, WNT16, XCL1, XCL2, XCR1, XCR1, XEDAR, XIAP, XPD, and receptors for hormones and growth factors. The genetically engineered fusion molecules of the present invention may bind one antigen or multiple antigens.

Preferred tumor targeting moieties contemplated for use in the fusion molecules of the present invention include depleting antibodies to specific tumor antigens, including, but not limited to, anti-Her2/neu, anti-Her3, anti-Her4, anti-CD20, anti-CD19, anti-CD22, anti-CXCR3, anti-CXCR5, anti-CCR3, anti-CCR4, anti-CCR9, anti-CRTH2, anti-PMCH, anti-CD4, and anti-CD25. All such tumor and inflammatory cell-specific, depleting antibodies have been well described in the literature.

The term “costimulatory molecule”, as used herein, is intended to refer to a group of immune cell surface receptor/ligands which engage between T cells and antigen presenting cells and generate a stimulatory signal in T cells which combines with the stimulatory signal (i.e., “co-stimulation”) in T cells that results from T cell receptor (“TCR”) recognition of antigen on antigen presenting cells, i.e., its art recognized meaning in immune T cell activation. Also contemplated for use are soluble forms of costimulatory molecules, i.e., those costimulatory molecules normally expressed by B cells, macrophages, monocytes, dendritic cells and other such antigen presenting cells. Costimulatory molecules contemplated for use thus include, but are not limited to, one or more of B7.1, B7.2, B7RP1, B7h, PD1, PDL1/PDL2, OX40L, CD86, CD40/CD40L or 41BB/41BBL. The choice of which costimulatory molecule to include in a particular embodiment depends upon, e.g., which particular immune response effects are desired, e.g., a humoral response, or a cellular immune response, or both. In certain embodiments both cellular and humoral immune responses against a disease related antigen are desired, and fusion molecules with varying costimulatory molecule domains are contemplated for use. Because of the nature of the targeting moiety, focused delivery of the signal is expected primarily to the tumor site. With focused delivery and dose optimization, the fusion molecules of the present invention are not expected to cause systemic activation of immune system leading to autoimmunity as seen with some non-antigen specific molecules currently in the clinical trials.

Chemokines are a superfamily of small (approximately about 4 to about 14 kDa), inducible and secreted pro-inflammatory cytokines that act primarily as chemoattractants and activators of specific leukocyte cell subtypes. Their production is induced by inflammatory cytokines, growth factors and pathogenic stimuli. Chemokine signaling results in the transcription of target genes involved in motility, cell invasion, and interactions with the extracellular matrix (ECM). Migration of cells that express the appropriate chemokine receptor occurs along the concentration gradient of the ligand known as the chemokine gradient; moving from a lower to higher concentration. Structural analysis demonstrates that most chemokines function as monomers and that the two regions necessary for receptor binding reside within the first 35 amino acids of the flexible N-terminus (Clark-Lewis et al., J Leukoc Biol. 57:703-11, 1995; Beall et al., Biochem J 313:633-40, 1996).

The chemokine proteins are divided into subfamilies based on conserved amino acid sequence motifs and are classified into four highly conserved groups—CXC, CC, C and CX3C, based on the position of the first two cysteines that are adjacent to the amino terminus. To date, more than 50 chemokines have been discovered and there are at least 18 human seven-transmembrane-domain (7TM) chemokine receptors. In general, these receptors, which belong to the G-protein-coupled receptor (GPCR) family, often bind to more than one type of chemokine. There are six non-promiscuous receptor-ligand pairs known to date—CXCR4-SDF1, CXCR5-CXCL13, CXCR6-CXCL16, CCR6-CCL20, CCR9-CCL25 (also known as TECK) and CX3CR1-CX3CL1 (also known as fractalkine or FKN).

The alpha subfamily (the CXC chemokines) has one amino acid separating the first two cysteine residues. The receptors for this group are designated CXCR1 to CXCR6. In the beta subfamily (the CC chemokines) the first two cysteines are adjacent to one another with no intervening amino acid. There are currently 24 distinct human beta subfamily members. The receptors for this group are designated CCR1 to CCR11. Target cells for different CC family members include most types of leukocytes. In the gamma subfamily (C chemokine), the chemokine protein contains two cysteines, corresponding to the first and third cysteines in the other groups. Lymphotactin is the lone member of the gamma class. The lymphotactin receptor is designated XCR1. In the delta subfamily (CX3C chemokine), the protein has three intervening amino acid between the first two cysteine residues. Fractalkine (FKN) is the only known member of the delta class. The fractalkine receptor is known as CX3CR1.

In a particularly preferred embodiment of the present invention, the chemokine will be FKN. This molecule is unique among chemokines in that it is a transmembrane protein with the N-terminal chemokine domain fused to a long mucin-like stalk. This membrane-anchored localization of FKN has led to the suggestion that it functions as a cell adhesion molecule for circulating inflammatory cells. Data supporting this hypothesis have come from numerous in vitro experiments showing that immobilized FKN, either on glass substrata or monolayers of CX3CR1 transfected cells, can support the capture and adhesion of leukocytes. These adhesive functions of FKN appear to be mediated by a single GPCR, CX3CRI, expressed on monocytes, DCs, NK cells, neurons, microglia and effector T-cells. In addition to functioning as an adhesion molecule, FKN can be released from the cell surface by a protease such as TACE(ADAM17) to generate a soluble molecule that has chemotactic activity for cells bearing the CX3CR1 receptor. By engineering a molecule with specificity to antigen (e.g. tumor antigen or a molecule overexpressed in pathological state) genetically fused with a chemokine ligand (full length, mutated for enhanced or dominant negative activity, truncated as to act for enhanced or dominant negative activity, or modified for enhanced or dominant negative activity) such as FKN will create chemokine gradient around tumor and therefore allow chemokine receptor expressing cells will migrate to tumor. Among many chemokine receptors, CX3CR1 is highly expressed on NK cells and effector cytotoxic T cells. Both of these cell types are rich in molecules needed for cell death, i.e. perforin and granzyme. By bringing NK cells and effector cytotoxic T cells closer to tumor through FKN we expect the following activities: (i) increased ADCC (ii) costimulation and thus adequate activation of effector cytotoxic T cells (iii) efficacy in patients with FcR mutation, i.e., retreatment opportunities for patients who previously failed to respond to antibody monotherapy. Other chemokines contemplated for use include, but are not limited to, MIP1a and MIP1.

In certain embodiments of the present invention, either the N- or C-terminus of antibody heavy or light chain will be genetically constructed with one of the several contemplated costimulatory ligands and/or chemokine. In other embodiments of the present invention, a targeting peptide or cytokine may be added to unused N- or C-terminus of antibody heavy or light chain to further enhance recruited cells activation in a tissue targeted fashion. The term “cytokine” is a generic term for proteins released by one cell population, which act on another cell population as intercellular mediators. Examples of such cytokines are lymphokines, monokines, and traditional polypeptide hormones. Included among the cytokines are growth hormone such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-alpha and -beta; mullerian-inhibiting substance; mouse gonadotropin-associated peptide; inhibin; activin; vascular endothelial growth factor; integrin; thrombopoietin (TPO); nerve growth factors such as NGF-alpha; platelet-growth factor; transforming growth factors (TGFs) such as TGF-alpha and TGF-beta; insulin-like growth factor-1 and -11; erythropoietin (EPO); osteoinductive factors; interferons such as interferon-alpha, -beta and -gamma colony stimulating factors (CSFs) such as macrophage-CSF (M-CSF); granulocyte macrophage-CSF (GM-CSF); and granulocyte-CSF (G-CSF); interleukins (ILs) such as IL-1, IL-2, IL-3, IL-4, IL-5, IL-6, IL-7, IL-8, IL-9, IL-10, IL-11, IL-12, IL-13, IL-15, IL-18, IL-23; a tumor necrosis factor such as TNF-alpha or TNF-beta; and other polypeptide factors including LIF and kit ligand (KL). As used herein, the term cytokine includes proteins from natural sources or from recombinant cell culture and biologically active equivalents of the native sequence cytokines.

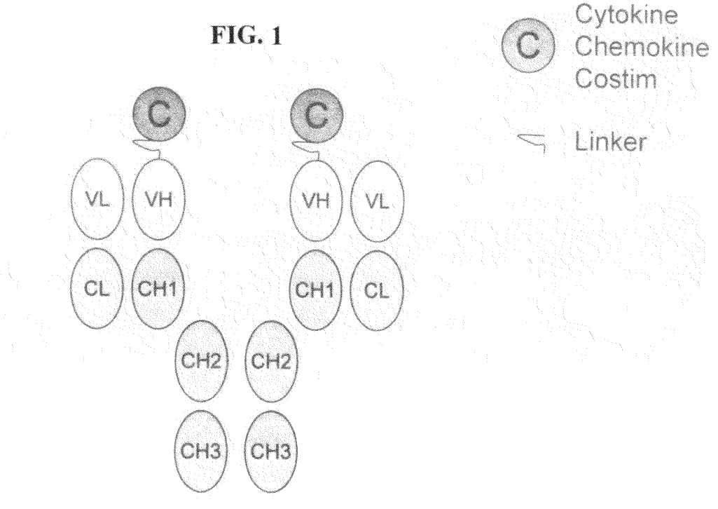

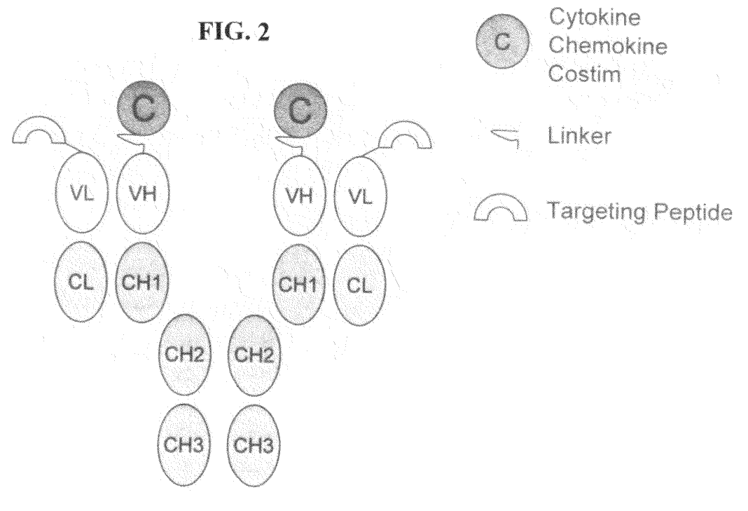

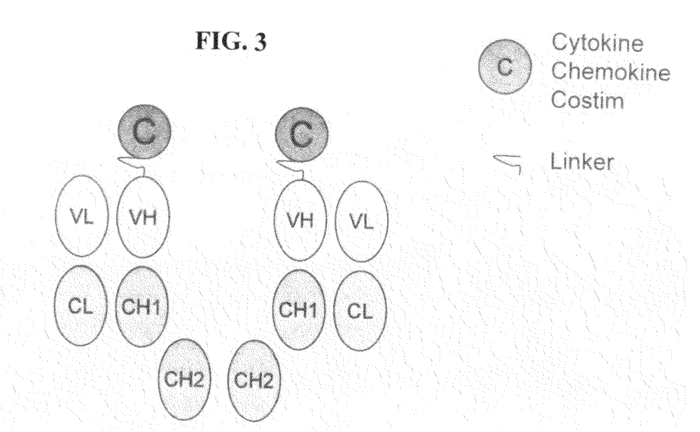

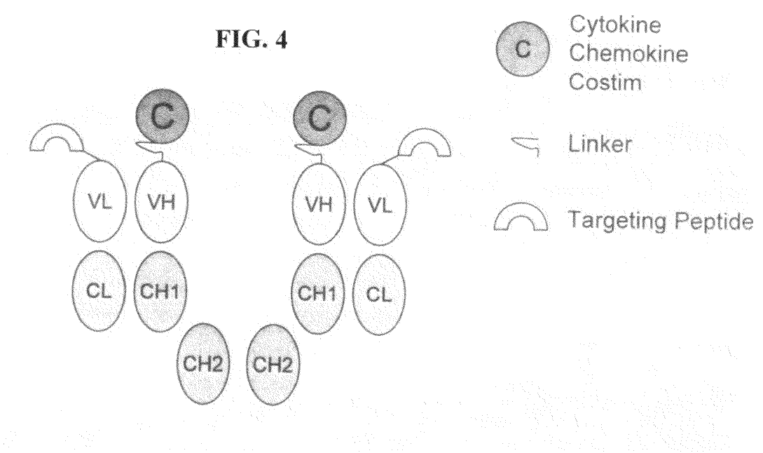

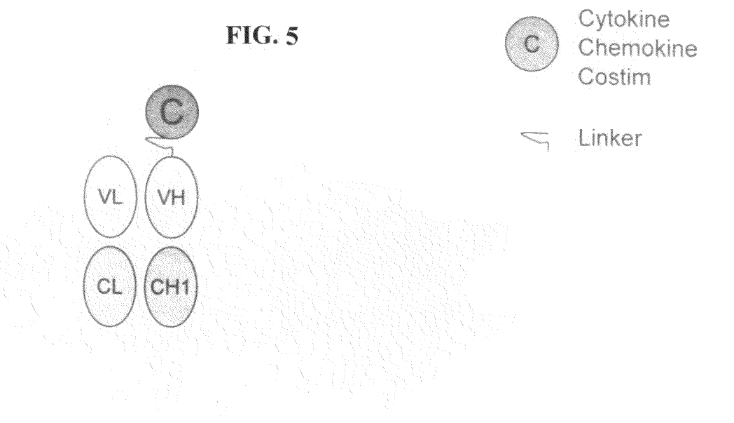

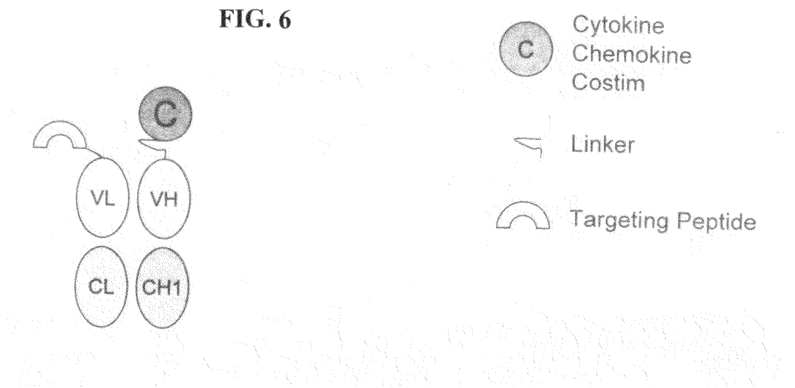

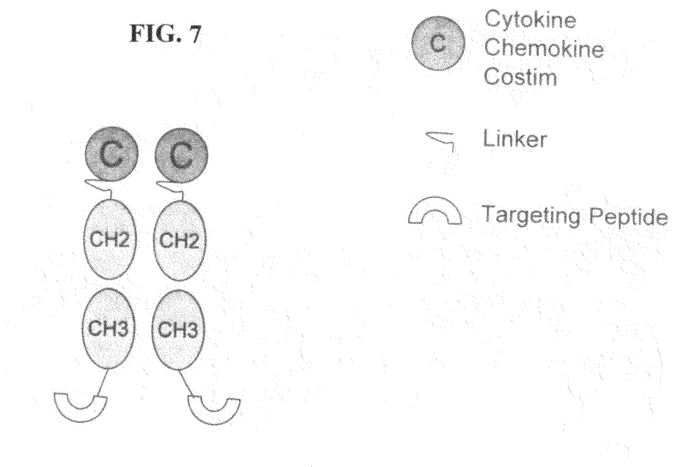

The genetically engineered fusion molecules utilized in the current invention are constructed using techniques well known to those of ordinary skill in the art. The fusion molecule may have the general designs as depicted in FIGS. 1-7. The method of preparing the fusion molecules generally comprises the steps of: 1) preparing/obtaining a cell/tumor targeting moiety; 2) preparing/obtaining a costimulatory molecule and/or a chemokine and/or cytokine; 3) preparing/obtaining a linker; 4) attaching 1) to 2) using said linker to prepare a fusion molecule; and 5) purifying said fusion molecule. Alternatively, the method may comprise, after step 4), step 5) preparing/obtaining a targeting peptide; step 6) preparing/obtaining a second linker; step 7) attaching the targeting peptide of step 5) to the fusion molecule of step 4) using said second linker to prepare a fusion molecule; and 8) purifying said fusion molecule.

For example, in one embodiment of the present invention, nucleic acid sequences encoding the appropriate tumor targeting moiety framework are optionally cloned and ligated into appropriate vectors (e.g., expression vectors for, e.g., prokaryotic or eukaryotic organisms). Additionally, nucleic acid sequences encoding the appropriate costimulatory molecule (or chemokine) are optionally cloned into the same vector in the appropriate orientation and location so that expression from the vector produces an tumor targeting moiety-costimulatory molecule (or chemokine) fusion molecule. Some optional embodiments also require post-expression modification, e.g., assembly of antibody subunits, etc. The techniques and art for the above (and similar) manipulations are well known to those skilled in the art. Pertinent instructions are found in, e.g., Sambrook et al., Molecular Cloning—A Laboratory Manual (2nd Ed.), Vols. 1-3, Cold Spring Harbor Laboratory, Cold Spring Harbor, N.Y., 1989 and Current Protocols in Molecular Biology, F. M. Ausubel et al., eds., Current Protocols, a joint venture between Greene Publishing Associates, Inc. and John Wiley & Sons, Inc. (supplemented through 1999).

Cells suitable for replicating and for supporting recombinant expression of protein are well known in the art. Such cells may be transfected or transduced as appropriate with the particular expression vector and large quantities of vector containing cells can be grown for seeding large scale fermenters to obtain sufficient quantities of the protein for clinical applications. Such cells may include prokaryotic microorganisms, such as E. coli; various eukaryotic cells, such as Chinese hamster ovary cells (CHO), NSO, 292; Yeast; insect cells; and transgenic animals and transgenic plants, and the like. Standard technologies are known in the art to express foreign genes in these systems.

The pharmaceutical compositions of the present invention comprise a genetically engineered fusion molecule of the invention and a pharmaceutically acceptable carrier. As used herein, “pharmaceutically acceptable carrier” means any and all solvents, dispersion media, coatings, antibacterial and antifungal agents, isotonic and absorption delaying agents, and the like that are physiologically compatible. Some examples of pharmaceutically acceptable carriers are water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Additional examples of pharmaceutically acceptable substances are wetting agents or minor amounts of auxiliary substances such as wetting or emulsifying agents, preservatives or buffers, which enhance the shelf life or effectiveness of the antibody. Except insofar as any conventional excipient, carrier or vehicle is incompatible with the genetically engineered fusion molecules of the present invention, its use in the pharmaceutical preparations of the invention is contemplated.

As used herein, the term “administration” refers to the act of giving a drug, prodrug, or other agent, or therapeutic treatment (e.g., radiation therapy) to a physiological system (e.g., a subject or in vivo, in vitro, or ex vivo cells, tissues, and organs). The compositions of this invention may be in a variety of forms, for example, liquid, semi-solid and solid dosage forms, such as liquid solutions (e.g., injectable and infusible solutions), dispersions or suspensions, tablets, pills, powders, liposomes and suppositories. A pharmaceutical composition of the invention is formulated to be compatible with its intended route of administration and therapeutic application. Methods of administering the pharmaceutical compositions of the present invention are via any route capable of delivering the composition to a tumor cell and include, but are not limited to, intradermal, intramuscular, intraperitoneal, intravenous, intratumor, subcutaneous, and the like. As will be appreciated by the skilled artisan, the route and/or mode of administration will vary depending upon the desired results. Typical preferred pharmaceutical compositions are in the form of injectable or infusible solutions, such as compositions similar to those used for passive immunization of humans. In a preferred embodiment, the composition is administered by intravenous infusion or injection. In another preferred embodiment, the composition is administered by intramuscular or subcutaneous injection.

The fusion molecules of the present invention and pharmaceutical compositions comprising them, can be administered in combination with one or more other therapeutic, diagnostic or prophylactic agents. Additional therapeutic agents include other anti-neoplastic, anti-tumor, anti-angiogenic or chemotherapeutic agents. Such additional agents may be included in the same composition or administered separately.

Therapeutic pharmaceutical compositions typically must be sterile and stable under the conditions of manufacture and storage. Sterile injectable solutions can be prepared by incorporating the fusion molecule in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

In certain embodiments, the pharmaceutical compositions active compounds may be prepared with a carrier that will protect the composition against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems (J. R. Robinson, ed., Marcel Dekker, Inc., New York, 1978).

In certain embodiments, the fusion molecules of the invention can be orally administered, for example, with an inert diluent or an assimilable edible carrier. The compound (and other ingredients, if desired) can also be enclosed in a hard or soft shell gelatin capsule, compressed into tablets, or incorporated directly into the subject's diet. For oral therapeutic administration, the fusion molecules can be incorporated with excipients and used in the form of ingestible tablets, buccal tablets, troches, capsules, elixirs, suspensions, syrups, wafers, and the like. To administer a compound of the invention by other than parenteral administration, it may be necessary to coat the compound with, or co-administer the compound with, a material to prevent its inactivation.

Additional active compounds also can be incorporated into the pharmaceutical compositions of the present invention. In certain embodiments, the fusion molecule of the invention is co-formulated with and/or co-administered with one or more additional therapeutic agents. These agents include, without limitation, antibodies that bind other targets, antineoplastic agents, antitumor agents, chemotherapeutic agents, and/or other agents known in the art that can enhance an immune response against tumor cells, e.g., IFN-.beta.1, IL-2, IL-8, IL-12, IL-15, IL-18, IL-23, IFN-.gamma., and GM-CSF. Such combination therapies may require lower dosages of the fusion molecule as well as the co-administered agents, thus avoiding possible toxicities or complications associated with the various monotherapies.

The pharmaceutical compositions of the invention may include a “therapeutically effective amount” or a “prophylactically effective amount” of the fusion molecule of the invention. As employed herein, the phrase “an effective amount,” refers to a dose sufficient to provide concentrations high enough to impart a beneficial effect on the recipient thereof. The specific therapeutically effective dose level for any particular subject will depend upon a variety of factors including the disorder being treated, the severity of the disorder, the activity of the specific compound, the route of administration, the rate of clearance of the compound, the duration of treatment, the drugs used in combination or coincident with the compound, the age, body weight, sex, diet, and general health of the subject, and like factors well known in the medical arts and sciences. Various general considerations taken into account in determining the “therapeutically effective amount” are known to those of skill in the art and are described, e.g., in Gilman et al., eds., Goodman And Gilman's: The Pharmacological Bases of Therapeutics, 8th ed., Pergamon Press, 1990; and Remington's Pharmaceutical Sciences, 17th ed., Mack Publishing Co., Easton, Pa., 1990. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

A therapeutically effective dose can be estimated initially from cell culture assays by determining an IC50. A dose can then be formulated in animal models to achieve a circulating plasma concentration range that includes the IC50 as determined in cell culture. Such information can be used to more accurately determine useful doses in humans. Levels in plasma may be measured, for example, by HPLC. The exact formulation, route of administration and dosage can be chosen by the individual physician in view of the patient's condition.

Dosage regimens can be adjusted to provide the optimum desired response (e.g., a therapeutic or prophylactic response). For example, a single bolus can be administered, several divided doses can be administered over time or the dose can be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the present invention will be dictated primarily by the unique characteristics of the tumor targeting moiety and the particular therapeutic or prophylactic effect to be achieved.

An exemplary, non-limiting range for a therapeutically or prophylactically effective amount of an antibody or antibody portion of the invention is 0.025 to 50 mg/kg, more preferably 0.1 to 50 mg/kg, more preferably 0.1-25, 0.1 to 10 or 0.1 to 3 mg/kg. It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

The genetically engineered fusion molecules of the present invention are useful in treating various diseases wherein the targets that are recognized by the molecules are detrimental. Such diseases include, but are not limited to, rheumatoid arthritis, osteoarthritis, juvenile chronic arthritis, septic arthritis, Lyme arthritis, psoriatic arthritis, reactive arthritis, spondyloarthropathy, systemic lupus erythematosus, Crohn's disease, ulcerative colitis, inflammatory bowel disease, insulin dependent diabetes mellitus, thyroiditis, asthma, allergic diseases, psoriasis, dermatitis scleroderma, graft versus host disease, organ transplant rejection, acute or chronic immune disease associated with organ transplantation, sarcoidosis, atherosclerosis, disseminated intravascular coagulation, Kawasaki's disease, Grave's disease, nephrotic syndrome, chronic fatigue syndrome, Wegener's granulomatosis, Henoch-Schoenlein purpurea, microscopic vasculitis of the kidneys, chronic active hepatitis, uveitis, septic shock, toxic shock syndrome, sepsis syndrome, cachexia, infectious diseases, parasitic diseases, acquired immunodeficiency syndrome, acute transverse myelitis, Huntington's chorea, Parkinson's disease, Alzheimer's disease, stroke, primary biliary cirrhosis, hemolytic anemia, malignancies, heart failure, myocardial infarction, Addison's disease, sporadic, polyglandular deficiency type I and polyglandular deficiency type II, Schmidt's syndrome, adult (acute) respiratory distress syndrome, alopecia, alopecia greata, seronegative arthopathy, arthropathy, Reiter's disease, psoriatic arthropathy, ulcerative colitic arthropathy, enteropathic synovitis, chlamydia, yersinia and salmonella associated arthropathy, spondyloarthopathy, atheromatous disease/arteriosclerosis, atopic allergy, autoimmune bullous disease, pemphigus vulgaris, pemphigus foliaceus, pemphigoid, linear IgA disease, autoimmune haemolytic anaemia, Coombs positive haemolytic anaemia, acquired pernicious anaemia, juvenile pernicious anaemia, myalgic encephalitis/Royal Free Disease, chronic mucocutaneous candidiasis, giant cell arteritis, primary sclerosing hepatitis, cryptogenic autoimmune hepatitis, Acquired Immunodeficiency Disease Syndrome, Acquired Immunodeficiency Related Diseases, Hepatitis B, Hepatitis C, common varied immunodeficiency (common variable hypogammaglobulinaemia), dilated cardiomyopathy, female infertility, ovarian failure, premature ovarian failure, fibrotic lung disease, cryptogenic fibrosing alveolitis, post-inflammatory interstitial lung disease, interstitial pneumonitis, connective tissue disease associated interstitial lung disease, mixed connective tissue disease associated lung disease, systemic sclerosis associated interstitial lung disease, rheumatoid arthritis associated interstitial lung disease, systemic lupus erythematosus associated lung disease, dermatomyositis/polymyositis associated lung disease, Sjogren's disease associated lung disease, ankylosing spondylitis associated lung disease, vasculitic diffuse lung disease, haemosiderosis associated lung disease, drug-induced interstitial lung disease, fibrosis, radiation fibrosis, bronchiolitis obliterans, chronic eosinophilic pneumonia, lymphocytic infiltrative lung disease, postinfectious interstitial lung disease, gouty arthritis, autoimmune hepatitis, type-1 autoimmune hepatitis (classical autoimmune or lupoid hepatitis), type-2 autoimmune hepatitis (anti-LKM antibody hepatitis), autoimmune mediated hypoglycaemia, type B insulin resistance with acanthosis nigricans, hypoparathyroidism, acute immune disease associated with organ transplantation, chronic immune disease associated with organ transplantation, osteoarthrosis, primary sclerosing cholangitis, psoriasis type 1, psoriasis type 2, idiopathic leucopaenia, autoimmune neutropaenia, renal disease NOS, glomerulonephritides, microscopic vasulitis of the kidneys, lyme disease, discoid lupus erythematosus, male infertility idiopathic or NOS, sperm autoimmunity, multiple sclerosis (all subtypes), sympathetic ophthalmia, pulmonary hypertension secondary to connective tissue disease, Goodpasture's syndrome, pulmonary manifestation of polyarteritis nodosa, acute rheumatic fever, rheumatoid spondylitis, Still's disease, systemic sclerosis, Sjorgren's syndrome, Takayasu's disease/arteritis, autoimmune thrombocytopaenia, idiopathic thrombocytopaenia, autoimmune thyroid disease, hyperthyroidism, goitrous autoimmune hypothyroidism (Hashimoto's disease), atrophic autoimmune hypothyroidism, primary myxoedema, phacogenic uveitis, primary vasculitis, vitiligo acute liver disease, chronic liver diseases, alcoholic cirrhosis, alcohol-induced liver injury, choleosatatis, idiosyncratic liver disease, Drug-Induced hepatitis, Non-alcoholic Steatohepatitis, allergy and asthma, group B streptococci (GBS) infection, mental disorders (e.g., depression and schizophrenia), Th2 Type and Th1 Type mediated diseases, acute and chronic pain (different forms of pain), and cancers such as lung, breast, stomach, bladder, colon, pancreas, ovarian, prostate and rectal cancer and hematopoietic malignancies (leukemia and lymphoma), Abetalipoprotemia, Acrocyanosis, acute and chronic parasitic or infectious processes, acute leukemia, acute lymphoblastic leukemia (ALL), acute myeloid leukemia (AML), acute or chronic bacterial infection, acute pancreatitis, acute renal failure, adenocarcinomas, aerial ectopic beats, AIDS dementia complex, alcohol-induced hepatitis, allergic conjunctivitis, allergic contact dermatitis, allergic rhinitis, allograft rejection, alpha-1-antitrypsin deficiency, amyotrophic lateral sclerosis, anemia, angina pectoris, anterior horn cell degeneration, anti cd3 therapy, antiphospholipid syndrome, anti-receptor hypersensitivity reactions, aordic and peripheral aneuryisms, aortic dissection, arterial hypertension, arteriosclerosis, arteriovenous fistula, ataxia, atrial fibrillation (sustained or paroxysmal), atrial flutter, atrioventricular block, B cell lymphoma, bone graft rejection, bone marrow transplant (BMT) rejection, bundle branch block, Burkitt's lymphoma, Burns, cardiac arrhythmias, cardiac stun syndrome, cardiac tumors, cardiomyopathy, cardiopulmonary bypass inflammation response, cartilage transplant rejection, cerebellar cortical degenerations, cerebellar disorders, chaotic or multifocal atrial tachycardia, chemotherapy associated disorders, chromic myelocytic leukemia (CML), chronic alcoholism, chronic inflammatory pathologies, chronic lymphocytic leukemia (CLL), chronic obstructive pulmonary disease (COPD), chronic salicylate intoxication, colorectal carcinoma, congestive heart failure, conjunctivitis, contact dermatitis, cor pulmonale, coronary artery disease, Creutzfeldt-Jakob disease, culture negative sepsis, cystic fibrosis, cytokine therapy associated disorders, Dementia pugilistica, demyelinating diseases, dengue hemorrhagic fever, dermatitis, dermatologic conditions, diabetes, diabetes mellitus, diabetic ateriosclerotic disease, Diffuse Lewy body disease, dilated congestive cardiomyopathy, disorders of the basal ganglia, Down's Syndrome in middle age, drug-induced movement disorders induced by drugs which block CNS dopamine receptors, drug sensitivity, eczema, encephalomyelitis, endocarditis, endocrinopathy, epiglottitis, epstein-barr virus infection, erythromelalgia, extrapyramidal and cerebellar disorders, familial hematophagocytic lymphohistiocytosis, fetal thymus implant rejection, Friedreich's ataxia, functional peripheral arterial disorders, fungal sepsis, gas gangrene, gastric ulcer, glomerular nephritis, graft rejection of any organ or tissue, gram negative sepsis, gram positive sepsis, granulomas due to intracellular organisms, hairy cell leukemia, Hallerrorden-Spatz disease, hashimoto's thyroiditis, hay fever, heart transplant rejection, hemachromatosis, hemodialysis, hemolytic uremic syndrome/thrombolytic thrombocytopenic purpura, hemorrhage, hepatitis (A), His bundle arrythmias, HIV infection/HIV neuropathy, Hodgkin's disease, hyperkinetic movement disorders, hypersensitity reactions, hypersensitivity pneumonitis, hypertension, hypokinetic movement disorders, hypothalamic-pituitary-adrenal axis evaluation, idiopathic Addison's disease, idiopathic pulmonary fibrosis, antibody mediated cytotoxicity, Asthenia, infantile spinal muscular atrophy, inflammation of the aorta, influenza a, ionizing radiation exposure, iridocyclitis/uveitis/optic neuritis, ischemia-reperfusion injury, ischemic stroke, juvenile rheumatoid arthritis, juvenile spinal muscular atrophy, Kaposi's sarcoma, kidney transplant rejection, legionella, leishmaniasis, leprosy, lesions of the corticospinal system, lipedema, liver transplant rejection, lymphederma, malaria, malignamt Lymphoma, malignant histiocytosis, malignant melanoma, meningitis, meningococcemia, metabolic/idiopathic, migraine headache, mitochondrial multi.system disorder, mixed connective tissue disease, monoclonal gammopathy, multiple myeloma, multiple systems degenerations (Mencel Dejerine-Thomas Shi-Drager and Machado-Joseph), myasthenia gravis, mycobacterium avium intracellulare, mycobacterium tuberculosis, myelodyplastic syndrome, myocardial infarction, myocardial ischemic disorders, nasopharyngeal carcinoma, neonatal chronic lung disease, nephritis, nephrosis, neurodegenerative diseases, neurogenic I muscular atrophies, neutropenic fever, non-hodgkins lymphoma, occlusion of the abdominal aorta and its branches, occulsive arterial disorders, okt3 therapy, orchitis/epidydimitis, orchitis/vasectomy reversal procedures, organomegaly, osteoporosis, pancreas transplant rejection, pancreatic carcinoma, paraneoplastic syndrome/hypercalcemia of malignancy, parathyroid transplant rejection, pelvic inflammatory disease, perennial rhinitis, pericardial disease, peripheral atherlosclerotic disease, peripheral vascular disorders, peritonitis, pernicious anemia, pneumocystis carinii pneumonia, pneumonia, POEMS syndrome (polyneuropathy, organomegaly, endocrinopathy, monoclonal gammopathy, and skin changes syndrome), post perfusion syndrome, post pump syndrome, post-MI cardiotomy syndrome, preeclampsia, Progressive supranucleo Palsy, primary pulmonary hypertension, radiation therapy, Raynaud's phenomenon and disease, Raynoud's disease, Refsum's disease, regular narrow QRS tachycardia, renovascular hypertension, reperfusion injury, restrictive cardiomyopathy, sarcomas, scleroderma, senile chorea, Senile Dementia of Lewy body type, seronegative arthropathies, shock, sickle cell anemia, skin allograft rejection, skin changes syndrome, small bowel transplant rejection, solid tumors, specific arrythmias, spinal ataxia, spinocerebellar degenerations, streptococcal myositis, structural lesions of the cerebellum, Subacute sclerosing panencephalitis, Syncope, syphilis of the cardiovascular system, systemic anaphalaxis, systemic inflammatory response syndrome, systemic onset juvenile rheumatoid arthritis, T-cell or FAB ALL, Telangiectasia, thromboangitis obliterans, thrombocytopenia, toxicity, transplants, trauma/hemorrhage, type III hypersensitivity reactions, type IV hypersensitivity, unstable angina, uremia, urosepsis, urticaria, valvular heart diseases, varicose veins, vasculitis, venous diseases, venous thrombosis, ventricular fibrillation, viral and fungal infections, vital encephalitis/aseptic meningitis, vital-associated hemaphagocytic syndrome, Wernicke-Korsakoff syndrome, Wilson's disease, xenograft rejection of any organ or tissue. (see Peritt et al. PCT publication No. WO2002097048A2, Leonard et al., PCT publication No. WO9524918 A1, and Salfeld et al., PCT publication No. WO00/56772A1).

Genetically engineered fusion molecules capable of binding the following pairs of targets to treat oncological disease are also contemplated: IGF1 and IGF2; IGF1/2 and Erb2B; VEGFR and EGFR; CD20 and CD3, CD138 and CD20, CD38 and CD20, CD38 & CD138, CD40 and CD20, CD138 and CD40, CD38 and CD40. Other target combinations include one or more members of the EGF/erb-2/erb-3 family. Other targets (one or more) involved in oncological diseases that DVD Igs may bind include, but are not limited to those selected from the group consisting of: CD52, CD20, CD19, CD3, CD4, CD8, BMP6, IL12A, IL1A, IL1B, IL2, IL24, INHA, TNF, TNFSF10, BMP6, EGF, FGF1, FGF10, FGF11, FGF12, FGF13, FGF14, FGF16, FGF17, FGF18, FGF19, FGF2, FGF20, FGF21, FGF22, FGF23, FGF3, FGF4, FGF5, FGF6, FGF7, FGF8, FGF9, GRP, IGF1, IGF2, IL12A, IL1A, IL1B, IL2, INHA, TGFA, TGFB1, TGFB2, TGFB3, VEGF, CDK2, EGF, FGF10, FGF18, FGF2, FGF4, FGF7, IGF1, IGF1R, IL2, VEGF, BCL2, CD164, CDKN1A, CDKN1B, CDKN1C, CDKN2A, CDKN2B, CDKN2C, CDKN3, GNRH1, IGFBP6, IL1A, IL1B, ODZ1, PAWR, PLG, TGFB1I1, AR, BRCA1, CDK3, CDK4, CDK5, CDK6, CDK7, CDK9, E2F1, EGFR, ENO1, ERBB2, ESR1, ESR2, IGFBP3, IGFBP6, IL2, INSL4, MYC, NOX5, NR6A1, PAP, PCNA, PRKCQ, PRKD1, PRL, TP53, FGF22, FGF23, FGF9, IGFBP3, IL2, INHA, KLK6, TP53, CHGB, GNRH1, IGF1, IGF2, INHA, INSL3, INSL4, PRL, KLK6, SHBG, NR1D1, NR1H3, NR1I3, NR2F6, NR4A3, ESR1, ESR2, NR0B1, NR0B2, NR1D2, NR1H2, NR1H4, NR1I2, NR2C1, NR2C2, NR2E1, NR2E3, NR2F1, NR2F2, NR3C1, NR3C2, NR4A1, NR4A2, NR5A1, NR5A2, NR6A1, PGR, RARB, FGF1, FGF2, FGF6, KLK3, KRT1, APOC1, BRCA1, CHGA, CHGB, CLU, COL1A1, COL6A1, EGF, ERBB2, ERK8, FGF1, FGF10, FGF11, FGF13, FGF14, FGF16, FGF17, FGF18, FGF2, FGF20, FGF21, FGF22, FGF23, FGF3, FGF4, FGF5, FGF6, FGF7, FGF8, FGF9, GNRH1, IGF1, IGF2, IGFBP3, IGFBP6, IL12A, IL1A, IL1B, IL2, IL24, INHA, INSL3, INSL4, KLK10, KLK12, KLK13, KLK14, KLK15, KLK3, KLK4, KLK5, KLK6, KLK9, MMP2, MMP9, MSMB, NTN4, ODZ1, PAP, PLAU, PRL, PSAP, SERPINA3, SHBG, TGFA, TIMP3, CD44, CDH1, CDH10, CDH19, CDH20, CDH7, CDH9, CDH1, CDH10, CDH13, CDH18, CDH19, CDH20, CDH7, CDH8, CDH9, ROBO2, CD44, ILK, ITGA1, APC, CD164, COL6A1, MTSS1, PAP, TGFB1I1, AGR2, AIG1, AKAP1, AKAP2, CANT1, CAV1, CDH12, CLDN3, CLN3, CYB5, CYC1, DAB21P, DES, DNCL1, ELAC2, ENO2, ENO3, FASN, FLJ12584, FLJ25530, GAGEB1, GAGEC1, GGT1, GSTP1, HIP1, HUMCYT2A, IL29, K6HF, KAI1, KRT2A, MIB1, PART1, PATE, PCA3, PIAS2, PIK3CG, PPID, PR1, PSCA, SLC2A2, SLC33A1, SLC43A1, STEAP, STEAP2, TPM1, TPM2, TRPC6, ANGPT1, ANGPT2, ANPEP, ECGF1, EREG, FGF1, FGF2, FIGF, FLT1, JAG1, KDR, LAMA5, NRP1, NRP2, PGF, PLXDC1, STAB1, VEGF, VEGFC, ANGPTL3, BAI1, COL4A3, IL8, LAMA5, NRP1, NRP2, STAB1, ANGPTL4, PECAM1, PF4, PROK2, SERPINF1, TNFAIP2, CCL1, CCL2, CXCL1, CXCL10, CXCL3, CXCL5, CXCL6, CXCL9, IFNA1, IFNB1, IFNG, IL1B, IL6, MDK, EDG1, EFNA1, EFNA3, EFNB2, EGF, EPHB4, FGFR3, HGF, IGF1, ITGB3, PDGFA, TEK, TGFA, TGFB1, TGFB2, TGFBR1, CCL2, CDH5, COL18A1, EDG1, ENG, ITGAV, ITGB3, THBS1, THBS2, BAD, BAG1, BCL2, CCNA1, CCNA2, CCND1, CCNE1, CCNE2, CDH1 (E-cadherin), CDKN1B (p27Kip1), CDKN2A (p6INK4a), COL6A1, CTNNB1 (b-catenin), CTSB (cathepsin B), ERBB2 (Her-2), ESR1, ESR2, F3 (TF), FOSL1 (FRA-1), GATA3, GSN (Gelsolin), IGFBP2, IL2RA, IL6, IL6R, IL6ST (glycoprotein 130), ITGA6 (a6 integrin), JUN, KLK5, KRT19, MAP2K7 (c-Jun), MKI67 (Ki-67), NGFB (NGF), NGFR, NME1 (NM23A), PGR, PLAU (uPA), PTEN, SERPINB5 (maspin), SERPINE1 (PAI-1), TGFA, THBS1 (thrombospondin-1), TIE (Tie-1), TNFRSF6 (Fas), TNFSF6 (FasL), TOP2A (topoisomerase Iia), TP53, AZGP1 (zinc-a-glycoprotein), BPAG1 (plectin), CDKN1A (p21Wap1/Cip1), CLDN7 (claudin-7), CLU (clusterin), ERBB2 (Her-2), FGF1, FLRT1 (fibronectin), GABRP (GABAa), GNAS1, ID2, ITGA6 (a6 integrin), ITGB4 (b 4 integrin), KLF5 (GC Box BP), KRT19 (Keratin 19), KRTHB6 (hair-specific type II keratin), MACMARCKS, MT3 (metallothionectin-III), MUC1 (mucin), PTGS2 (COX-2), RAC2 (p21Rac2), S100A2, SCGB1D2 (lipophilin B), SCGB2A1 (mammaglobin 2), SCGB2A2 (mammaglobin 1), SPRR1B (Spr1), THBS1, THBS2, THBS4, and TNFAIP2 (B94).

This invention also relates to pharmaceutical compositions for inhibiting abnormal cell growth in a mammal comprising an amount of a fusion molecule of the invention in combination with an amount of a chemotherapeutic, wherein the amounts of the fusion molecule and of the chemotherapeutic are together effective in inhibiting abnormal cell growth. Many chemotherapeutics are presently known in the art. In some embodiments, the chemotherapeutic is selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, anti-hormones, e.g. anti-androgens, and anti-angiogenesis agents.

The following examples are provided to describe the invention in further detail.

### Example 1

This Example describes the preparation of genetically engineered molecules comprising a tumor targeting moiety and a chemokine. In this example, the chemokine is fractalkine or MCP-1 and the tumor targeting moiety is an anti-her2/neu antibody. The molecule will be constructed as depicted in FIG. 1, with the fractalkine molecule (full length or truncated) or MCP-1 molecule attached via a linker to the heavy chain (HC)(e.g., IGN02/03/04/06 below) or light chain (LC)(e.g., IGN01 below) of the antibody. The molecules in this example were prepared using methods and techniques well known and understood by one of ordinary skill in the art.

The preparation of the engineered molecules can be generally described as follows: 1) the full gene sequence of interest is synthesized by the most appropriate method, e.g., direct gene synthesis, overlap PCR methodologies, and/or restriction-ligation techniques, for such gene; 2) the synthesized gene sequence is incorporated into an appropriate expression vector; 3) the expression vector is sequence verified; 4) an expression vector is constructed to be produced and purified in sufficient quantities from E. coli fermentation; 5) DNA plasmids are constructed and used to perform pilot transfections of HEK-293 Freestyle cells and the expression of the protein of interest is monitored using reagents and/or protocols capable of detecting the protein of interest; 6) using the information from the pilot transfections, production scale up is performed by transfection of HEK-293 Freestyle cells with DNA plasmids so as to produce sufficient conditioned medium from the transfected HEK-293 Freestyle cells to deliver the target amount of purified protein; 7) conditioned medium is collected, concentrated, and the protein of interest purified using a single Protein A affinity chromatography step or appropriate alternative chromatography methods; 8) the final product is formulated in a desired buffer and at a desired concentration (the protein concentration is confirmed by UV absorption); and 9) the purity of the final product is determined by SDS-PAGE.

The actual construction of six engineered molecules is depicted as follows:

| | |
|---|---|
| IGN01 | |
| Light chain | |
| QHHGVTKCNITCSKMTSKTPVALLIHYQQNQASCGKRAIILETRQHRLFCADPKEQWVK | (SEQ ID NO:31) |
| DAMQHLDRQAAALTRNGGGGGSGGGGSGGGGSDIQMTQSPSSLSASVGDRVTTTCRAS | |
| QDVNTAVAWYQQKPGKAPKLLIYSASFLYSGVPSRFSGSRSGTDFTLTISSLQPEDFATY | |
| YCQQHYTTPPTFGQGTKVEIKRTVAAPSVFIFPPSDEQLKSGTASVVCLLNNFYPREAKV | |
| QWKVDNALQSGNSQESVTEQDSKDSTYSLSSTLTLSKADYEKHKVYACEVTHQGLSSP | |
| VTKSFNRGEC | |
| Heavy chain | |
| EVQLVESGGGLVQPGGSLRLSCAASGFNIKDTYIHWVRQAPGKGLEWVARIYPTNGYT | (SEQ ID NO:32) |
| RYADSVKGRFTISADTSKNTAYLQMNSLRAEDTAVYYCSRWGGDGFYAMDYWGQGT | |
| LVTVSSASTKGPSVFPLAPSSKSTSGGTAALGCLVKDYFPEPVTVSWNSGALTSGVHTFP | |
| AVLQSSGLYSLSSVVTVPSSSLGTQTYICNVNHKPSNTKVDKKVEPPKSCDKTHTCPPCP | |
| APELLGGPSVFLFPPKPKDTLMISRTPEVTCVVVDVSHEDPEVKYNWYVDGVEWINAKT | |
| KPREEQYNSTYRVVSVLTVLHQDWLNGKEYKCKVSNKALPAPIEKTISKAKGQPREPQ | |
| VYTLPPSRDELTKNQVSLTCLVKGFYPSDIAVEWESNGQPENNYKTTPPVLDSDGSFFLY | |
| SKLTVDKSRWQQGNVFSCSVMHEALHNHYTQKSLSLSPGK | |

In the sequences above, the Light chain sequence would be representative of the C sequence (amino acids 1-76), the linker sequence (amino acids 77-91) and the VL and CL sequences (amino acids 92-305) in FIG. 1. In this example, the C sequence was the chemokine, fractalkine. The Heavy chain sequence would be representative of the VH, CH1, CH2, and CH3 sequences in FIG. 1.

| | |
|---|---|
| IGN02 | |
| Light chain | |
| DIQMTQSPSSLSASVGDRVTITCRASQDVNTAVAWYQQKPGKAPKLLIYSASFLYSGVP | (SEQ ID NO:33) |
| SRFSGSRSGTDFTLTISSLQPEDFATYYCQQHYTTPPTFGQGTKVEIKRTVAAPSVFIFPP | |
| SDEQLKSGTASVVCLLNNFYPREAKVQWKVDNALQSGNSQESVTEQDSKDSTYSLSST | |
| LTLSKADYEKHKVYACEVTHQGLSSPVTKSFNRGEC | |
| Heavy chain | |
| QHHGVTKCNITCSKMTSKIPYALLLHYQQNQASCGKRAIILETRQHRLFCADPKEQWVK | (SEQ ID NO:34) |
| DAMQHLDRQAAALTRNGGGGGSGGGGSGGGGSEVQLVESGGGLVQPGGSLRLSCAAS | |
| GFNIKDTYLHWVRQAPGKGLEWVARIYPTNGYTRYADSVKGRFTLSADTSKNTAYLQM | |
| NSLRAEDTAVYYCSRWGGDGFYAMDYWGQGTLVTVSSASTKGPSVFPLAPSSKSTSGG | |
| TAALGCLVKDYFPEPVTVSWNSGALTSGVHTFPAVLQSSGLYSLSSVVTVPSSSLGTQT | |
| YICNVNHKPSNTKVDKKVEPPKSCDKTHTCPPCPAPELLGGPSVFLFPPKPKDTLMISRTP | |
| EVTCVVVDVSHEDPEVKFNWYVDGVEVHNAKTKPREEQYNSTYRVVSVLTVLHQDWL | |
| NGKEYKCKVSNKALPAPLEKTISKAKGQPREPQVYTLPPSRDELTKNQVSLTCLVKGFYP | |
| SDIAVEWESNGQPENNYKTTPPVLDSDGSFFLYSKLTVDKSRWQQGNVFSCSVMHEAL | |
| HNHYTQKSLSLSPGK | |

In the sequences above, the Light chain sequence would be representative of the VL and CL sequences in FIG. 1. The Heavy chain sequence would be representative of the C sequence (amino acids 1-76), the linker sequence (amino acids 77-91) and the VH, CH1, CH2, and CH3 sequences (amino acids 92-542) in FIG. 1. In this example, the C sequence was the chemokine, fractalkine.

| | |
|---|---|
| IGN03 | |
| Light chain | |
| DIQMTQSPSSLSASVGDRVTITCRASQDVNTAVAWYQQKPGKAPKLLIYSASFLYSGVP | (SEQ ID NO:33) |
| SRFSGSRSGTDFTLTISSLQPEDFATYYCQQHYTTPPTFGQGTKVEIKRTVAAPSVFIFPP | |
| SDEQLKSGTASVVCLLNNFYPREAKVQWKVDNALQSGNSQESVTEQDSKDSTYSLSST | |
| LTLSKADYEKHKVYACEVTHQGLSSPVTKSFNRGEC | |
| Heavy chain | |
| EVQLVESGGGLVQPGGSLRLSCAASGFNIKDTYIHWVRQAPGKGLEWVARIYPTNGYT | (SEQ ID NO:35) |
| RYADSVKGRFTISADTSKNTAYLQMNSLRAEDTAVYYCSRWGGDGFYAMDYWGQGT | |
| LVTVSSASTKGPSVFPLAPSSKSTSGGTAALGCLVKDYFPEPVIVSWNSGALTSGVHTFP | |
| AVLQSSGLYSLSSVVTVPSSSLGTQTYICNVNHKPSNTKVDKKVEPPKSCDKTHTCPPCP | |
| APELLGGPSVFLFPPKPKDTLMISRTPEVTCVVVDVSHEDPEVKFNWYVDGVEVHNAKT | |
| KPREEQYNSTYRVVSVLTVLHQDWLNGKEYKCKVSNKALPAPIEKTISKAKGQPREPQ | |
| VYTLPPSRDELTKNQVSLTCLVKGFYPSDLAVEWESNGQPENNYKTITPPVLDSDGSFFLY | |
| SKLTVDKSRWQQGNVFSCSVMHEALHNHYTQKSLSLSPGGGGGSGGGGSGGGGSQHH | |
| GVTKCNITCSKMTSKIPVALLIHYQQNQASCGKRAIILETRQHRLFCADPKEQWVKDAM | |
| QHLDRQAAALTRNG | |

In the sequences above, the Light chain sequence would be representative of the VL and CL sequences in FIG. 1. The Heavy chain sequence would be representative of the VH, CH1, CH2, and CH3 sequences (amino acids 1450), the linker sequence (amino acids 451-465) and the C sequence (amino acids 466-541) in FIG. 1. In this example, the C sequence was the chemokine, fractalkine.

| | |
|---|---|
| IGN04 | |
| Light chain | |
| DIQMTQSPSSLSASVGDRVT1TCRASQDVNTAVAWYQQKPGKAPKLL1YSASFLYSGVP | (SEQ ID NO:33) |
| SRFSGSRSGTDFTLTISSLQPEDFATYYCQQHYTTPPTFGQGTKVEJKRTVAAPSVFLFPP | |
| SDEQLKSGTASVVCLLNNFYPREAKVQWKVDNALQSGNSQESVTEQDSKDSTYSLSST | |
| LTLSKADYEKHXVYACEVTHQGLSSPVTKSFNRGEC | |
| Heavy chain | |
| QHHGVTKCNITCSKMTSKIPVALLIHYQQNQASCGKRAIILETRQHRLFCADPKEQWVK | (SEQ ID NO:36) |
| DAMQHLDRQAAALTRNGGGGGSGGGGSGGGGSGGGGSGGGGSEVQLVESGGGLVQP | |
| GGSLRLSCAASGFNIKDTYIHWVRQAPGKGLEWVARIYPTNGYTRYADSVKGRFTISAD | |
| TSKNTAYLQMNSLRAEDTAVYYCSRWGGDGFYAMDYWGQGTLVTVSSASTKGPSVFP | |
| LAPSSKSTSGGTAALGCLVKDYFPEPVTVSWNSGALTSGVHTFPAVLQSSGLYSLSSVVT | |
| VPSSSLGTQTYICNVNHKPSNTKVDKKVEPPKSCDKTHTCPPCPAPELLGGPSVFLFPPKP | |
| KDTLMISRTPEVTCVVVDVSHEDPEVKFNWYVDGVEVHNAKTKPREEQYNSTYRVVS | |
| VLTVLHQDWLNGKEYKCKVSNKALPAPIEKTLSKAKGQPREPQVYTLPPSRDELTKNQV | |
| SLTCLVKGFYPSDIAVEWESNGQPENNYKTTPPVLDSDGSFFLYSKLTVDKSRWQQGNV | |
| FSCSVMHEALHNHYTQKSLSLSPGK | |

In the sequences above, the Light chain sequence would be representative of the VL and CL sequences in FIG. 1. The Heavy chain sequence would be representative of the C sequence (amino acids 1-76), the linker sequence (amino acids 77-101) and the VH, CH1, CH2, and CH3 sequences (amino acids 102-552) in FIG. 1. In this example, the C sequence was the chemokine, fractalkine.

### IGN05

The molecule tested as IGN05 was a synthetically prepared protein having a sequence similar to that of Herceptin® (Genentech USA). IGN05 contained no linker sequence or C sequence.

| | |
|---|---|
| IGN06 | |
| Light chain | |
| DIQMTQSPSSLSASVGDRVTITCRASQDVNTAVAWYQQKPGKAPKLLIYSASFLYSGVP | (SEQ ID NO:33) |
| SRFSGSRSGTDFTLTISSLQPEDFATYYCQQHYTTPPTFGQGTKVEIKRTVAAPSVFIFPP | |
| SDEQLKSGTASVVCLLNNFYPREAKVQWKVDNALQSGNSQESVTEQDSKDSTYSLSST | |
| LTLSKADYEKHKVYACEVTHQGLSSPVTKSFNRGEC | |
| Heavy chain | |
| QPDAINAPVTCCYNFTNRKISVQRLASYRRITSSKCPKEAVIFKTIVAKEICADPKQKWV | (SEQ ID NO:37) |
| QDSMDHLDKQTQTPKTGGGGSGGGGSGGGGSGGGGSGGGGSEVQLVESGGGLVQPGG | |
| SLRLSCAASGFNIKDTYIHWVRQAPGKGLEWVARIYPTNGYTRYADSVKGRFTISADTS | |
| KNTAYLQMNSLRAEDTAVYYCSRWGGDGFYAMDYWGQGTLVTVSSASTKGPSVFPL | |
| APSSKSTSGGTAALGCLVKDYFPEPVTVSWNSGALTSGVHTFPAVLQSSGLYSLSSVVT | |
| VPSSSLGTQTYICNVNHKPSNTKVDKKVEPPKSCDKTHTCPPCPAPELLGGPSVFLFPPKP | |
| KDTLMISRTPEVTCVVVDVSHEDPEVKFNWYVDGVEVHNAKTKPREEQYNSTYRVVS | |
| VLTVLHQDWLNGKEYKCKVSNKALPAPIEKTISKAKGQPREPQVYTLPPSRDELTKNQV | |
| SLTCLVKGFYPSDLAVEWESNGQPENNYKTTPPVLDSDGSFFLYSKLTVDKSRWQQGNV | |
| FSCSVMHEALHNHYTQKSLSLSPGK | |

In the sequences above, the Light chain sequence would be representative of the VL and CL sequences in FIG. 1. The Heavy chain sequence would be representative of the C sequence (amino acids 1-76), the linker sequence (amino acids 77-101) and the VH, CH1, CH2, and CH3 sequences (amino acids 102-552) in FIG. 1. In this example, the C sequence was the chemokine, MCP-1.

### Example 2

In this Example, the molecules of Example 1 were tested and evaluated head-to-head with commercially available Herceptin® in a SKBR-3 breast cancer cell binding assay and in an ADCC assay.

### Cell Binding

Binding of the IGN01-06 molecules to SKBR-3 breast cancer cells was measured by two methods. In the first method, a labeled antibody to CX3CL1(fractalkine)(R&D Systems Cat. No. IC365P) was used. By binding to the fractalkine moiety of the fusion proteins, this antibody acted as a secondary antibody for the detection of the cell-bound antibody of interest. In the second method, a labeled anti-human IgG (Jackson ImmunResearch Cat. No. 109-096-088) was used to bind to the IgG moiety of the fusion proteins. Both of these antibodies were used at a fixed concentration of 5 ug/ml.

The results of these studies are shown below. Binding curves for anti-IgG and CX3CL1 are shown in FIGS. 8 and 9, respectively. The relative binding is shown in Table 1. The values in FIGS. 8 and 9 are given as mean fluorescence intensity (MFI), which represents the average fluorescence of the cells measured by FACS as a function of antibody concentration. In Table 1, the values for maximal binding have been normalized to the control (cells stained with secondary antibody alone) to obtain the fold-increase in fluorescence relative to the control.

All antibody variants bound to SKBR3 cells, as indicated by the fact that the anti-IgG secondary antibody labeled the cells at all of the concentrations evaluated (FIG. 8). Compared to IGN05 binding to SKBR3 cells, IGN02, 03, 04, and 06 was 75-80% (FIG. 8, Table 1). The binding of IGN01 was 29% of IGN05. With respect to the binding of anti-CX3CL1 antibody, IGN05 and 06 failed to bind (shown in FIG. 9). Because neither IGN05 nor IGN06 have a CX3CL1 domain, they served as negative control in this assay. The antibody bound to IGN03 to a moderate degree (50-fold above background) while the remaining variants bound 10- to 20-fold above background.

TABLE 1
Table 1. Maximal antibody binding to SKBR3
cells treated with IGN01-06 molecules.

| | CX3CL1 | CX3CL1 | IgG | TgG |
|---|---|---|---|---|
| Variant | MFI | Relative MFI | MFI | Relative MFI |
| 1 | 84.5 | 10.8 | 130.7 | 32.7 |
| 2 | 100.4 | 12.9 | 352.0 | 88.0 |
| 3 | 404.3 | 51.8 | 340.6 | 85.2 |
| 4 | 151.9 | 19.4 | 306.4 | 76.6 |
| 5 | 7.8 | 1.0 | 445.2 | 111.3 |
| 6 | 8.0 | 1.0 | 343.1 | 85.8 |
| Herceptin ® | — | — | 448.0 | 112.0 |
| Ctrl | 7.8 | 1.0 | 4 | 1.0 |

The mean fluorescence intensity (MFI) and relative MFI (MFI normalized to control) at a saturating concentration of variant (1 ug/ml) are shown for anti-CX3CL1 and anti-IgG antibodies.

### ADCC Assay

The IGN01-06 were also test in an ADCC assay. PBMC-mediated ADCC against SKBR3 cells was measured using FACS. Briefly, target cells were labeled with carboxyfluorescein, treated with IGN01-06 variant constructs, mixed with PBMCs at an E:T of 50:1 and incubated for 4 hours at 37° C. At that time, propidium iodide was added to label dead cells and the samples were analyzed by FACS. Using such a methodology, live cells fluoresce green while dead target cells fluoresce both green and red. Importantly, each of the IGN01-06 molecules demonstrated ADCC activity with the EC₅₀ ranging 0.039-0.1552 μg/ml, indicating that the addition of chemokine ligand did not cause steric hinderance to effect ADCC.

The engineered IGN01-04 and IGN06 molecules have two sites to interact with effector cells: 1) Fc interaction with Fc receptors; and 2) chemokine interaction with chemokine receptor. As relates to the fractalkine-containing molecules, the fractalkine receptor, CX3CR1, has been shown to express in most NK and effector cytotoxic T cells. Moreover, fractalkine will help in the migration of the cells to closer proximity of the tumor thus providing efficient recruitment of NK and effector cytotoxic T cells. It is possible that fractalkine-containing molecules may overcome killer-inhibitory receptor mediated protection in the growth of some tumors. As such, these chemokine-containing molecules will demonstrate superior tumor killing activity of tumor cells as compared to Herceptin® alone, including efficacy in patients refractory to previous therapies due to Fc receptor polymorphisms in cancer patients, thereby improving on existing tumor antigen-specific, depleting antibody therapies.

### Example 3

This Example describes the preparation of a genetically engineered molecule comprising a tumor targeting moiety and a costimulatory molecule. In this example, the costimulatory molecules is OX40L, 41BBL, or CD86 and the tumor targeting moiety is an anti-her2/neu antibody. The molecule will be constructed as depicted in FIG. 1, with the costimulatory molecule (full length or truncated) attached via a linker to the heavy chain (VH) of the antibody. The molecule will be prepared using the methods described herein. The molecule will be tested and evaluated in a cell based assay involving tumor cells and T cells and wherein said tumor cell can not normally costimulate or activate T cells. Importantly, because the tumor targeting moiety of the engineered molecule will bind the tumor and display the costimulatory molecule on the surface of the tumor, the molecule will effectively activate T cells and help in killing the tumor, thereby improving on current antigen-specific cytotoxic T cells mediated therapies.

All of the articles and methods disclosed and claimed herein can be made and executed without undue experimentation in light of the present disclosure. While the articles and methods of this invention have been described in terms of preferred embodiments, it will be apparent to those of skill in the art that variations may be applied to the articles and methods without departing from the spirit and scope of the invention. All such variations and equivalents apparent to those skilled in the art, whether now existing or later developed, are deemed to be within the spirit and scope of the invention as defined by the appended claims. All patents, patent applications, and publications mentioned in the specification are indicative of the levels of those of ordinary skill in the art to which the invention pertains. All patents, patent applications, and publications are herein incorporated by reference in their entirety for all purposes and to the same extent as if each individual publication was specifically and individually indicated to be incorporated by reference in its entirety for any and all purposes. The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. A genetically engineered fusion molecule comprising a tumor targeting moiety attached to a chemokine, wherein said fusion molecule exhibits increased ADCC/tumor killing and enhanced activation of T cells at the tumor site as compared to said tumor target moiety.

2. A fusion molecule of claim 1 wherein said tumor target moiety is an antibody.

3. A fusion molecule of claim 2 wherein said antibody is an anti-her2/neu antibody.

4. A fusion molecule of claim 3, wherein said chemokine is fractalkine.

5. A fusion molecule of claim 3, wherein said chemokine is MCP-1.

6. A fusion molecule of claim 1, wherein said tumor target moiety is attached to said chemokine by a linker.

7. A fusion molecule of claim 1, further comprising a targeting peptide fused to the tumor target moiety.

8. A pharmaceutical composition comprising a fusion molecule of claim 1 in a pharmaceutically acceptable carrier.

9. A method for modulating an immune response in a patient, comprising: administering to said patient a therapeutically effective amount of a pharmaceutical composition of claim 8.

10. A method for treating tumors or tumor metastases in a patient, comprising: administering to said patient a therapeutically effective amount of a pharmaceutical composition of claim 8.

11. A genetically engineered fusion molecule comprising a tumor targeting moiety attached to one or more costimulatory molecules, wherein said fusion molecule delivers said costimulatory molecule to the tumor site and promotes activation of T cells at the tumor site.

12. A fusion molecule of claim 11, further comprising a targeting peptide fused to the tumor target moiety.
